(19) United States
(12) Patent Application Publication
(10) Pub. No.: US 2024/0327470 A1
(43) Pub. Date: Oct. 3, 2024

(54) MODIFIED MENINGOCOCCAL FHBP POLYPEPTIDES

(71) Applicant: GLAXOSMITHKLINE BIOLOGICALS SA, Rixensart (BE)

(72) Inventors: Matthew BOTTOMLEY, Siena (IT); Vega MASIGNANI, Siena (IT)

(73) Assignee: GLAXOSMITHKLINE BIOLOGICALS SA, Rixensart (BE)

(21) Appl. No.: 18/438,870

(22) Filed: Feb. 12, 2024

Related U.S. Application Data
(63) Continuation of application No. 17/346,362, filed on Jun. 14, 2021, now Pat. No. 11,939,357.
Application No. 17/346,362 is a continuation of application No. 15/319,651, filed on Dec. 16, 2016, now Pat. No. 11,066,450.

(30) Foreign Application Priority Data
Jul. 17, 2014 (EP) 14177564.3

(51) Int. Cl. C07K 14/22; A61K 39/00; A61K 39/095

(52) U.S. Cl. CPC C07K 14/22; A61K 39/095; A61K 2039/575; C07K 2319/33

(57) ABSTRACT

The inventors have identified residues within variant 2 and variant 3 of meningococcal fHpb which can be modified to enhance their properties.

### CROSS-REFERENCE TO RELATED APPLICATIONS

This is a Divisional of U.S. patent application Ser. No. 17/346,362, filed Jun. 14, 2021, which is a Continuation of U.S. patent application Ser. No. 15/319,651, filed Dec. 16, 2016, now issued as U.S. Pat. No. 11,066,450 on Jul. 20, 2021, which is the U.S. National Stage Application submitted under 35 U.S.C. § 371 of International Application No. PCT/EP2015/066228, filed Jul. 16, 2015, which claims priority to European Patent Application No. EP 14177564.3, filed on Jul. 17, 2014, all of which are incorporated herein by reference in their entireties.

### REFERENCE TO SEQUENCE LISTING

The instant application contains a Sequence Listing which has been submitted electronically in XML file format and is hereby incorporated by reference in its entirety. Said XML copy, created on Feb. 6, 2024, is named VN56307D1-US_SL.xml and is 96,487 bytes in size.

### TECHNICAL FIELD

This invention is in the field of protein engineering, relating in particular to the meningococcal factor H binding protein (fHbp), which is known to be a useful vaccine immunogen.

### BACKGROUND

Neisseria meningitidis is a Gram-negative encapsulated bacterium which colonises the upper respiratory tract of approximately 10% of human population. Conjugate vaccines are available against serogroups A, C, W135 and Y, but the only vaccine which is available for protecting against serogroup B in general is the BEXSERO (vaccine) product which was approved in 2013.

One of the protective immunogens in BEXSERO (vaccine) is fHbp, which has also been known as protein ‘741’ (SEQ ID NO: 2536 in ref. 1; SEQ ID 1 herein), ‘NMB1870’, ‘GNA1870’ [2-4, ‘P2086’, ‘LP2086’ or ‘ORF2086’ [5-7]. The 3D structure of this protein is known [8,9], and the protein has two β-barrels connected by a short linker. Many publications have reported on the protective efficacy of this protein in meningococcal vaccines e.g. see references 10-14. The fHbp lipoprotein is expressed in various strains across all serogroups. fHbp sequences have been grouped into three variants [2](referred to herein as v1, v2 and v3), and it has been found in general that serum raised against a given variant is bactericidal against strains which express that variant, but is not active against strains which express one of the other two variants i.e. there is intra-variant cross-protection, but not inter-variant cross-protection (except for some v2 and v3 cross-reactivity).

To increase inter-family cross-reactivity the fHbp sequence has been engineered to contain specificities for all three variants [15]. Protein engineering has also been used to remove fHbp's interaction with siderophores [16] and with human factor H [17-25]. Disruption of the interaction with fH has been reported for all three variants and is postulated to provide a superior vaccine immunogen [22,26]. For v2 polypeptides, however, references 23 and 24 report an inherent instability which is also seen in mutants with disrupted fH-binding. The instability appears to arise from the N-terminal β-barrel domain, and reference 23 warns that any substitutions in this barrel might promote instability. Mutations which aim to improve the stability of v2 sequences are disclosed in reference 27.

It is an object of the invention to provide fHbp v2 and v3 mutants having enhanced properties.

### SUMMARY OF THE INVENTION

In one aspect, the present invention provides a polypeptide comprising an amino acid sequence having at least 80% sequence identity to SEQ ID NO: 5, wherein the amino acid sequence differs from SEQ ID NO: 5 at residues 123 and 240 relative to SEQ ID NO:5, and the polypeptide can, after administration to a human, elicit antibodies which can recognise a polypeptide consisting of SEQ ID NO: 4.

Another aspect of the invention is a polypeptide comprising an amino acid sequence having at least 80% sequence identity to SEQ ID NO: 5, wherein the amino acid sequence differs from SEQ ID NO: 5 at residues 32, 123 and 240 relative to SEQ ID NO:5, and the polypeptide can, after administration to a human, elicit antibodies which can recognise a polypeptide consisting of SEQ ID NO: 4.

Another aspect of the invention is a polypeptide comprising an amino acid sequence having at least 80% sequence identity to SEQ ID NO: 17, wherein the amino acid sequence differs from SEQ ID NO: 17 at residues 126 and 243 relative to SEQ ID NO: 17, and the polypeptide can, after administration to a human, elicit antibodies which can recognise a polypeptide consisting of SEQ ID NO: 40.

Another aspect of the invention is a polypeptide comprising an amino acid sequence having at least 80% sequence identity to SEQ ID NO: 17, wherein the amino acid sequence differs from SEQ ID NO: 17 at residues 32, 126 and 243 relative to SEQ ID NO: 17, and the polypeptide can, after administration to a human, elicit antibodies which can recognise a polypeptide consisting of SEQ ID NO: 40.

Another aspect of the invention is a polypeptide comprising an amino acid sequence having at least 90% sequence identity to SEQ ID NO: 47, wherein relative to SEQ ID NO:47 residue 123 is not leucine and residue 240 is not glutamate; and wherein when administered to a human the polypeptide can elicit an antibody response that is bactericidal against a meningococcus which expresses a v2 fHbp.

Another aspect of the invention is a polypeptide comprising an amino acid sequence having at least 90% sequence identity to SEQ ID NO: 47, wherein relative to SEQ ID NO:47 residue 32 is not serine, residue 123 is not leucine and residue 240 is not glutamate; and wherein when administered to a human the polypeptide can elicit an antibody response that is bactericidal against a meningococcus which expresses a v2 fHbp.

Another aspect of the invention is a polypeptide comprising an amino acid sequence having at least 90% sequence identity to SEQ ID NO: 48 wherein relative to SEQ ID NO:48 residue 126 is not leucine and residue 243 is not glutamate; and wherein when administered to a human the polypeptide can elicit an antibody response that is bactericidal against a meningococcus which expresses a v3 fHbp.

Another aspect of the invention is a polypeptide comprising an amino acid sequence having at least 90% sequence identity to SEQ ID NO: 48 wherein relative to SEQ ID NO:48 residue 32 is not serine, residue 126 is not leucine and residue 243 is not glutamate; and wherein when administered to a human the polypeptide can elicit an antibody response that is bactericidal against a meningococcus which expresses a v3 fHbp.

Another aspect of the invention is fusion polypeptides which, when administered to a human, elicit an antibody response that is bactericidal against both a meningococcus expressing a v2 fHbp and a meningococcus expressing a v3 fHbp.

Another aspect of the invention is an immunogenic composition comprising a pharmaceutically acceptable carrier and a polypeptide or fusion polypeptide of the invention.

Another aspect of the invention is a method of raising an antibody response in a human, comprising administering to said human an immunogenic composition according to the invention.

### BRIEF DESCRIPTION OF THE DRAWINGS

FIG. 1 shows the SPR response for wild-type v2 fHbp (top line), the E266 mutant of v2 (bottom line) and S58V/L149R mutant of v2 (middle line) binding to immobilised fH. The y-axis shows relative units, and the x-axis shows time (seconds, with 0 being sample injection).

FIG. 2 shows DSC results for wild-type and S58V/L149R mutant v2 fHbp. The C-terminal domain was unaffected by the mutation, but the Tm of the N-terminal domain was increased by ≥20° C. (marked with the arrow). The y-axis shows Cp (kcal/mol/° C.), and the x-axis shows temperature (° C.).

FIG. 3 shows DSC results for wild-type and E266A mutant v2 fHbp. The N-terminal transition vanishes in the mutant, but the Tm of the C-terminal domain was increased by ≥16° C.

FIG. 4 shows the SPR response of the ‘wild-type’ (upper line, dashed) and ‘SNB mutant’ (lower line, solid) v2-v3-v1 fHbp fusion polypeptides.

FIG. 5A and FIG. 5B show DSC results for the (FIG. 5A) ‘wild-type’ and (FIG. 5B) ‘SNB mutant’ v2-v3-v1 fHbp fusion polypeptides.

FIG. 6 shows the SPR response of v3 fHbp, either as wild-type (top) or with various mutations.

FIG. 7A and FIG. 7B show the structures of v2 (FIG. 7A) and v3 (FIG. 7B) fHbp determined by X-ray crystallography in the absence of factor H following stabilisation of fHbp using the S58 and L149 mutations.

FIG. 8A and FIG. 8B compare stability of the ‘wild-type’ fusion (SEQ ID NO: 18) in comparison with the stabilised non-binding fusion (SEQ ID NO: 27) in E. coli extracts probed by Western blot. FIG. 8A shows that truncated forms of the stabilised non-binding fusion are less prevalent. FIG. 8B demonstrates that the stabilised non-binding fusion is less prone to cleavage by chymotrypsin than the ‘wild-type’ fusion.

FIG. 9 provides a schematic of the 231 SNB fusion.

### DETAILED DESCRIPTION

Full-length fHbp from strain 2996 in v2 has the following amino acid sequence (SEQ ID NO: 2):

MNRTAFCCLSLTAALILTACSSGGGGVAADIGAGLADALTAPLDHKDKS
LQSLTLDQSVRKNEKLKLAAQGAEKTYGNGDSLNTGKLKNDKVSRFDFI
RQIEVDGQLITLESGEFQIYKQDHSAVVALQIEKINNPDKIDSLINQRS
FLVSGLGGEHTAFNQLPDGKAEYHGKAFSSDDAGGKLTYTIDFAAKQGH
GKIEHLKTPEQNVELAAAELKADEKSHAVILGDTRYGSEEKGTYHLALF
GDRAQEIAGSATVKIGEKVHEIGIAGKQ

The mature lipoprotein lacks the first 19 amino acids of SEQ ID NO: 2 (underlined; provides SEQ ID NO: 4, beginning with Cys-20). It is also known to produce a ‘ΔG’ form of fHbp in which the N-terminus is truncated up to residue 26 (i.e. to remove the poly-glycine stretch, and beginning instead with Val-27), thus providing SEQ ID NO: 5.

Full-length fHbp from strain M1239 in v3 has the following amino acid sequence (SEQ ID NO: 3):

MNRTAFCCLSLTTALILTACSSGGGGSGGGGVAADIGTGLADALTAPLD
HKDKGLKSLTLEDSIPQNGTLTLSAQGAEKTFKAGDKDNSLNTGKLKND
KISRFDFVQKIEVDGQTITLASGEFQIYKQNHSAVVALQIEKINNPDKT
DSLINQRSFLVSGLGGEHTAFNQLPGGKAEYHGKAFSSDDPNGRLHYSI
DFTKKQGYGRIEHLKTLEQNVELAAAELKADEKSHAVILGDTRYGSEEK
GTYHLALFGDRAQEIAGSATVKIGEKVHEIGIAGKQ

The mature lipoprotein lacks the first 19 amino acids of SEQ ID NO: 3 (underlined; provides SEQ ID NO: 40), and the ΔG form of SEQ ID NO: 3 lacks the first 31 amino acids (SEQ ID NO: 17).

The inventors have studied two different types of mutation in v2 and v3. Firstly, they have identified residues within SEQ ID NO: 2 and SEQ ID NO: 3 which can be modified to increase the polypeptide's stability. Secondly, they have identified residues which decrease binding to human factor H (fH). The invention relates to mutant fHbp polypeptides comprising both types of mutation, thereby providing fHbp polypeptides with enhanced properties. Specifically, fHbp mutants that do not bind factor H but which retain immunogenicity are advantageous because the resultant antibody responses are directed towards epitopes in or near the fH-binding site. Following vaccination using wild-type fHbp vaccine antigens, such epitopes may be obscured by factor H binding.

The amino acids of most interest are as follows, numbered according to the full-length sequences (SEQ ID NOs: 1 & 3) and also according to the ΔG sequences (SEQ ID NOs: 5 & 17):

| | |
|---|---|
| Stability** | fH binding |

| | | | | |
|---|---|---|---|---|
| v2 | SEQ ID NO: 1 | Ser-58 | Leu-149 | Glu-266 |
| | SEQ ID NO: 5 | Ser-32 | Leu-123 | Glu-240 |
| v3 | SEQ ID NO: 3 | Ser-63 | Leu-157 | Glu-274 |
| | SEQ ID NO: 17 | Ser-32 | Leu-126 | Glu-243 |

**Where only one of these residues is mutated, it is preferably the leucine

Mutant v2 fHbp

Thus in a first aspect, the invention provides a polypeptide comprising a mutant fHbp v2 amino acid sequence, wherein: (a) the amino acid sequence has at least k % sequence identity to SEQ ID NO: 5, and/or comprises a fragment of SEQ ID NO: 5; but (b) the amino acid sequence differs from SEQ ID NO: 5 at residues L123 and E240 (and, optionally, at residue S32) (numbering of amino acids is relative to SEQ ID NO:5).

Where feature (a) relates to a fragment, the fragment will include the two (or optionally three) residues listed in (b), but those residues will differ when compared to those positions in SEQ ID NO: 5. A mutant fHbp v2 amino acid sequence can have at least k % sequence identity to, and include several fragments of, SEQ ID NO: 5, wherein each such fragment is at least 7 amino acids long. These fragments will typically include at least one epitope from SEQ ID NO: 5. Epitope identification and mapping is established for fHbp [11; 28-32].

The value of k may be selected from 80, 85, 86, 87, 88, 89, 90, 91, 92, 93, 94, 95, 96, 97, 98, 99 or more. It is preferably 90 (i.e. the mutant fHbp v2 amino acid sequence has at least 90% identity to SEQ ID NO: 5) and is more preferably 95.

The polypeptide can, after administration to a suitable host animal (such as a mouse or human), elicit antibodies which can recognise a wild-type meningococcal polypeptide consisting of SEQ ID NO: 4.

These antibodies will include some antibodies which do not recognise a v1 or a v3 polypeptide (e.g. will not recognise a wild-type meningococcal polypeptide consisting of SEQ ID NO: 46 and a wild-type meningococcal polypeptide consisting of SEQ ID NO: 40), although they may also include some antibodies which cross-react with v1 and/or v3 polypeptides. The antibodies are ideally bactericidal against a meningococcal strain which expresses a v2 fHbp e.g. against the M2091 strain (see below).

The polypeptide has, under the same experimental conditions, a higher stability than the same polypeptide but without the sequence differences of (b), e.g. higher stability than a wild-type meningococcal polypeptide consisting of SEQ ID NO: 4. The stability enhancement can be assessed using differential scanning calorimetry (DSC) e.g. as discussed in references 33 & 34. DSC has previously been used to assess the stability of v2 fHbp [24]. Suitable conditions for DSC to assess stability can use 20 μM of polypeptide in a buffered solution (e.g. 25 mM Tris) with a pH between 6 and 8 (e.g. 7-7.5) with 100-200 mM NaCl (e.g. 150 mM).

The increase in stability is ideally at least 5° C. e.g. at least 10° C., 15° C., 20° C., 25° C., 30° C., 35° C. or more. These temperatures refer to the increase in thermal transition midpoint (Tm) as assessed by DSC. Wild-type fHbp shows two DSC peaks during unfolding (one for the N-terminal domain and one for the C-terminal domain) and, where a polypeptide of the invention includes both such domains, the increase refers to the stability of the N-terminal domain, which can occur even below 40° C. with wild-type v2 sequences [24] (whereas C-terminal domains can have a Tm of 80° C. or more). Thus the mutant fHbp v2 amino acid sequence of the invention preferably has a N-terminal domain with a Tm of at least 45° C. e.g. ≥50° C., ≥55° C., ≥60° C., ≥65° C., ≥70° C., ≥75° C., or even ≥80° C.

In addition to this increased stability the polypeptide has, under the same experimental conditions, a lower affinity for human fH than the same polypeptide but without the sequence differences of (b), e.g. lower affinity than a wild-type meningococcal polypeptide consisting of SEQ ID NO: 4. The affinity disruption can be quantitatively assessed using surface plasmon resonance (SPR), e.g. as disclosed in references 18 and 21-24 with immobilised human fH. An affinity reduction (i.e. an increase in the dissociation constant, K_{D}) of at least 10-fold, and ideally at least 100-fold, is preferred.

In some embodiments, the polypeptide is truncated relative to SEQ ID NO: 5. Compared to the wild-type mature sequence, SEQ ID NO: 5 is already truncated at the N-terminus up to and including the poly-glycine sequence (compare SEQ ID NOs: 4 and 5), but SEQ ID NO: 5 can be truncated at the C-terminus and/or further truncated at the N-terminus.

Mutant v3 fHbp

In a second aspect, the invention provides a polypeptide comprising a mutant fHbp v3 amino acid sequence, wherein: (a) the amino acid sequence has at least j % sequence identity to SEQ ID NO: 17, and/or comprises a fragment of SEQ ID NO: 17; but (b) the amino acid sequence differs from SEQ ID NO: 17 at residues L126 and E243 (and, optionally, at residue S32) (numbering of amino acids is relative to SEQ ID NO:17.

Where feature (a) relates to a fragment, the fragment will include the two (or optionally three) residues listed in (b), but those residues will differ when compared to those positions in SEQ ID NO: 17. A mutant fHbp v3 amino acid sequence can have at least j % sequence identity to and include several fragments of SEQ ID NO: 17, wherein each such fragment is at least 7 amino acids long. These fragments will typically include at least one epitope from SEQ ID NO: 17. Epitope identification and mapping is established for fHbp [11; 28-32].

The value of j may be selected from 80, 85, 86, 87, 88, 89, 90, 91, 92, 93, 94, 95, 96, 97, 98, 99 or more. It is preferably 90 (i.e. the mutant fHbp v3 amino acid sequence has at least 90% identity to SEQ ID NO: 17) and is more preferably 95.

The polypeptide can, after administration to a suitable host animal (such as a mouse or human), elicit antibodies which can recognise a wild-type meningococcal polypeptide consisting of SEQ ID NO: 40. These antibodies will include some antibodies which do not recognise a v1 or a v2 polypeptide (e.g. will not recognise a wild-type meningococcal polypeptide consisting of SEQ ID NO: 46 and a wild-type meningococcal polypeptide consisting of SEQ ID NO: 4), although they may also include some antibodies which cross-react with v1 and/or v2 polypeptides. The antibodies are ideally bactericidal against a meningococcal strain which expresses a v3 fHbp e.g. against the M01-240355 strain (see below).

The polypeptide has, under the same experimental conditions, a higher stability than the same polypeptide but without the sequence differences of (b), e.g. higher stability than a wild-type meningococcal polypeptide consisting of SEQ ID NO: 40. The stability enhancement can be assessed using differential scanning calorimetry (DSC) e.g. as discussed in references 33 & 34 DSC has previously been used to assess the stability of v3 fHbp [23]. Suitable conditions for DSC to assess stability can use 20 μM of polypeptide in a buffered solution (e.g. 25 mM Tris) with a pH between 6 and 8 (e.g. 7-7.5) with 100-200 mM NaCl (e.g. 150 mM).

The increase in stability is ideally at least 5° C. e.g. at least 10° C., 15° C., 20° C., 25° C., 30° C., 35° C. or more. These temperatures refer to the increase in thermal transition midpoint (Tm) as assessed by DSC. Wild-type fHbp shows two DSC peaks during unfolding (one for the N-terminal domain and one for the C-terminal domain) and, where a polypeptide of the invention includes both such domains, the increase refers to the stability of the N-terminal domain, which can occur at around 60° C. or less with wild-type v3 sequences [24] (whereas C-terminal domains can have a Tm of 80° C. or more). Thus the mutant fHbp v3 amino acid sequence of the invention preferably has a N-terminal domain with a Tm of at least 65° C. e.g. ≥70° C., ≥75° C., or even ≥80° C.

In addition to this increased stability the polypeptide has, under the same experimental conditions, a lower affinity for human fH than the same polypeptide but without the sequence differences of (b), e.g. lower affinity than a wild-type meningococcal polypeptide consisting of SEQ ID NO: 40. The affinity disruption can be quantitatively assessed using surface plasmon resonance (SPR), e.g. as disclosed in references 18 and 21-24 with immobilised human fH. An affinity reduction (i.e. an increase in the dissociation constant, K_{D}) of at least 10-fold, and ideally at least 100-fold, is preferred.

In some embodiments, the polypeptide is truncated relative to SEQ ID NO: 17. Compared to the wild-type mature sequence, SEQ ID NO: 17 is already truncated at the N-terminus up to and including the poly-glycine sequence (compare SEQ ID NOs: 40 and 17), but SEQ ID NO: 17 can be truncated at the C-terminus and/or further truncated at the N-terminus.

### Mutations Relative to SEQ ID NO: 5

Polypeptides of the first aspect of the invention comprise an amino acid sequence which has at least k % identity to SEQ ID NO: 5, and/or comprise a fragment of SEQ ID NO: 5. In comparison to SEQ ID NO: 5, however, this amino sequence has a modification at least at amino acid residues L123 and E240 (and optionally also at residue S32). These residues are numbered according to SEQ ID NO: 5; to match the nascent wild-type sequence (SEQ ID NO: 2), the numbering should change +26 (i.e. Ser-32 of SEQ ID NO: 5 is Ser-58 of SEQ ID NO: 2), and to match the mature wild-type sequence (SEQ ID NO: 4) the numbering should change +7 (which also permits easy comparison with ref. 25).

The three specified residues can be deleted, but preferably they are substituted by a different amino acid. For example, Leu-123 can be substituted by any of the other 19 naturally-occurring amino acids. When a substitution is made, the replacement amino acid in some embodiments may be a simple amino acid such as glycine or alanine. In other instances, the replacement amino acid is a conservative substitution e.g. it is made within the following four groups: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. In other embodiments the substitution is non-conservative.

Preferred substitutions at the specified residues are as follows: S32V; L123R; and E240A.

In addition to the mutation(s) noted above, which aim to increase stability and disrupt the polypeptide's ability to bind to fH, a polypeptide can include one or more further mutation(s) e.g. to disrupt the polypeptide's interaction with siderophores. Residues which interact with siderophores can be mutated, using the guidance in references 16 and 35, e.g. by aligning SEQ ID NO: 5 herein with SEQ ID NO: 4 of reference 16 to identify residues which can interact with siderophores e.g. with catecholates, hydroxamates or carboxylates.

Reference 24 reports that certain substitutions in v2 can increase affinity for fH, and so these should usually be avoided e.g. E85 in SEQ ID NO: 5 (residue 157 in ref. 24).

Further residues can also be mutated provided that, compared to the wild-type sequence (e.g. SEQ ID NO: 4), the polypeptide has higher stability, has lower affinity for fH, and when administered to a suitable mammal can elicit an antibody response that is bactericidal against meningococcus.

The polypeptide of the first aspect can comprise SEQ ID NO: 47. In SEQ ID NO: 47, residue 32 is any amino acid, residue 123 is not leucine, and residue 240 is not glutamate. A further option is SEQ ID NO:39, where residue 32 is not serine, residue 123 is not leucine, and residue 240 is not glutamate. In a preferred embodiment of SEQ ID NO: 47, residue 32 is valine, residue 123 is arginine, and residue 240 is alanine (i.e. SEQ ID NO: 50). In another preferred embodiment of SEQ ID NO: 47, residue 32 is serine, residue 123 is arginine, and residue 240 is alanine (i.e. SEQ ID NO: 53).

The polypeptide of the first aspect can comprise SEQ ID NO: 31. In SEQ ID NO: 31, residue 32 is any amino acid, and residue 123 is not leucine. A further option is SEQ ID NO:37, where residue 32 is not serine, and residue 123 is not leucine. In a preferred embodiment of SEQ ID NO: 31, residue 32 is valine, and residue 123 is arginine (i.e. SEQ ID NO: 45). In another preferred embodiment of SEQ ID NO: 31, residue 32 is serine, and residue 123 is arginine (i.e. SEQ ID NO: 54).

The amino acid residues noted for mutation in a v2 sequence are numbered relative to SEQ ID NO: 5 which is from strain 2996. The corresponding amino acid residues in a v2 fHbp from any other strain can be readily identified by sequence alignment e.g. being the amino acid which, when aligned to SEQ ID NO: 5 using a pairwise alignment algorithm (e.g. the Needleman-Wunsch global alignment algorithm, as detailed below), aligns with the amino acid mentioned herein. Often the amino acid will be the same as seen in SEQ ID NO: 5 (e.g. residue 32 will be serine), but the alignment will easily identify if this is not the case.

### Mutations Relative to SEQ ID NO: 17

Polypeptides of the second aspect of the invention comprise an amino acid sequence which has at least j % identity to SEQ ID NO: 17, and/or comprise a fragment of SEQ ID NO: 17. In comparison to SEQ ID NO: 17, however, this amino sequence has a modification at least at amino acid residues L126 and E243 (and, optionally, at residue S32). These residues are numbered according to SEQ ID NO: 17; to match the nascent wild-type sequence (SEQ ID NO: 3), the numbering should change +31 (i.e. Ser-32 of SEQ ID NO: 17 is Ser-63 of SEQ ID NO: 3), and to match the mature wild-type sequence (SEQ ID NO: 40) the numbering should change +12.

The two (or three) specified residues can be deleted, but preferably they are substituted by a different amino acid. For example, Leu-126 can be substituted by any of the other 19 naturally-occurring amino acids. When a substitution is made, the replacement amino acid in some embodiments may be a simple amino acid such as glycine or alanine. In other embodiments, the replacement amino acid is a conservative substitution e.g. it is made within the following four groups: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. In other embodiments the substitution is non-conservative.

Preferred substitutions at the specified residues are as follows: S32V; L126R; and E243A.

In addition to the mutation(s) noted above, which aim to increase stability and disrupt the polypeptide's ability to bind to fH, a polypeptide can include one or more further mutation(s) e.g. to disrupt the polypeptide's interaction with siderophores. Residues which interact with siderophores can be mutated, using the guidance in references 16 and 35, e.g. by aligning SEQ ID NO: 17 herein with SEQ ID NO: 4 of reference 16 to identify residues which can interact with siderophores e.g. with catecholates, hydroxamates or carboxylates.

Reference 24 reports that certain substitutions in v3 can increase affinity for fH, and so these should usually be avoided e.g. P44 in SEQ ID NO: 17 (residue 106 in ref. 24).

Further residues can also be mutated provided that, compared to the wild-type sequence (e.g. SEQ ID NO: 40), the polypeptide has higher stability, has lower affinity for fH, and when administered to a suitable mammal can elicit an antibody response that is bactericidal against meningococcus.

The polypeptide of the second aspect can comprise SEQ ID NO: 48. In SEQ ID NO: 48, residue 32 is any amino acid, residue 126 is not leucine, and residue 243 is not glutamate. A further option is SEQ ID NO: 57, where residue 32 is not serine, residue 126 is not leucine, and residue 243 is not glutamate.

In a preferred embodiment of SEQ ID NO: 48, residue 32 is valine, residue 126 is arginine, and residue 243 is alanine (i.e. SEQ ID NO: 51). In another preferred embodiment of SEQ ID NO: 48, residue 32 is serine, residue 126 is arginine, and residue 243 is alanine (i.e. SEQ ID NO: 55).

The polypeptide of the second aspect can comprise SEQ ID NO: 32. In SEQ ID NO: 32, residue 32 is any amino acid, and residue 126 is not leucine. A further option is SEQ ID NO:38, where residue 32 is not serine, residue 126 is not leucine. In a preferred embodiment of SEQ ID NO: 32, residue 32 is valine, and residue 126 is arginine (i.e. SEQ ID NO: 44). In another preferred embodiment of SEQ ID NO: 32, residue 32 is serine, and residue 126 is arginine (i.e. SEQ ID NO: 56).

The amino acid residues noted for mutation in a v3 sequence are numbered relative to SEQ ID NO: 17 which is from strain M1239. The corresponding amino acid residues in a v3 fHbp from any other strain can be readily identified by sequence alignment e.g. being the amino acid which, when aligned to SEQ ID NO: 17 using a pairwise alignment algorithm (e.g. the Needleman-Wunsch global alignment algorithm, as detailed below), aligns with the amino acid mentioned herein. Often the amino acid will be the same as seen in SEQ ID NO: 17 (e.g. residue 32 will be serine), but the alignment will easily identify if this is not the case.

### Mutant Sequences of the Invention

As mentioned above, the polypeptide of the first aspect of the invention can comprise SEQ ID NO: 47 or SEQ ID NO: 37, and the polypeptide of the second aspect can comprise SEQ ID NO: 48 or SEQ ID NO:57.

In a third aspect of the invention, which overlaps with the first aspect, the invention provides a polypeptide comprising an amino acid sequence having at least v % sequence identity to SEQ ID NO: 47, provided that (i) residue 32 is any amino acid, but in some embodiments is not serine (ii) residue 123 is not leucine (iii) residue 240 is not glutamate (iv) compared to the wild-type sequence, e.g. SEQ ID NO: 4, the polypeptide has higher stability and has lower affinity for fH (v) when administered to a suitable mammal can elicit an antibody response that is bactericidal against a meningococcus which expresses a v2 fHbp. The residue numbering of (i) to (iii) is according to SEQ ID NO: 47.

The value of v may be selected from 80, 85, 86, 87, 88, 89, 90, 91, 92, 93, 94, 95, 96, 97, 98, 99 or more. It is preferably 90 (i.e. the mutant fHbp v2 amino acid sequence has at least 90% identity to SEQ ID NO: 47) and is more preferably 95.

In a fourth aspect of the invention, which overlaps with the second aspect, the invention provides a polypeptide comprising an amino acid sequence having at least w % sequence identity to SEQ ID NO: 48, provided that (i) residue 32 is any amino acid, but in some embodiments is not serine (ii) residue 126 is not leucine (iii) residue 243 is not glutamate (iv) compared to the wild-type sequence, e.g. SEQ ID NO: 40, the polypeptide has higher stability and has lower affinity for fH (v) when administered to a suitable mammal can elicit an antibody response that is bactericidal against a meningococcus which expresses a v3 fHbp. The residue numbering of (i) to (iii) is according to SEQ ID NO: 48.

The value of w may be selected from 80, 85, 86, 87, 88, 89, 90, 91, 92, 93, 94, 95, 96, 97, 98, 99 or more. It is preferably 90 (i.e. the mutant fHbp v3 amino acid sequence has at least 90% identity to SEQ ID NO: 48) and is more preferably 95.

In a fifth aspect, the invention provides a polypeptide comprising amino acid sequence SEQ ID NO: 47, modified by up to 5 single amino acid changes (i.e. 1, 2, 3, 4 or 5 single amino acid substitutions, deletions and/or insertions), provided that (i) residue 32 is any amino acid, but in some embodiments is not serine (ii) residue 123 is not leucine (iii) residue 240 is not glutamate (iv) compared to the wild-type sequence, e.g. SEQ ID NO: 4, the polypeptide has higher stability and has lower affinity for fH (v) when administered to a suitable mammal can elicit an antibody response that is bactericidal against a meningococcus which expresses a v2 fHbp.

In a sixth aspect, the invention provides a polypeptide comprising amino acid sequence SEQ ID NO: 48, modified by up to 5 single amino acid changes (i.e. 1, 2, 3, 4 or 5 single amino acid substitutions, deletions and/or insertions), provided that (i) residue 32 is any amino acid, but in some embodiments is not serine (ii) residue 126 is not leucine (iii) residue 243 is not glutamate (iv) compared to the wild-type sequence, e.g. SEQ ID NO: 40, the polypeptide has higher stability and has lower affinity for fH (v) when administered to a suitable mammal can elicit an antibody response that is bactericidal against a meningococcus which expresses a v3 fHbp. The residue numbering of (i) to (iii) is according to SEQ ID NO: 48.

These various v2 and v3 polypeptides can be combined in fusion polypeptides, thereby providing immune responses against both variants with a single polypeptide. Thus a seventh aspect of the invention provides a polypeptide comprising a fusion of: (i) a polypeptide as defined according to the first, third, or fifth aspects of the invention; and (ii) a polypeptide as defined according to the second, fourth, or sixth aspects of the invention. Advantageously, such fusion polypeptides can, when administered to a suitable mammal, elicit an antibody response that is bactericidal against both a meningococcus which expresses a v2 fHbp and a meningococcus which expresses a v3 fHbp.

Thus, within the seventh aspect, the fusion polypeptide comprises:
(I) a first amino acid sequence selected from:
   a mutant fHbp v2 amino acid sequence, wherein: (a) the amino acid sequence has at least k % sequence identity to SEQ ID NO: 5, and/or comprises a fragment of SEQ ID NO: 5; but (b) the amino acid sequence differs from SEQ ID NO: 5 at residues L123 and E240 (and, optionally, also at residue S32);
   an amino acid sequence having at least v % sequence identity to SEQ ID NO: 47, provided that (i) residue 32 is any amino acid, but in some embodiments is not serine (ii) residue 123 is not leucine (iii) residue 240 is not glutamate; or
   the amino acid sequence SEQ ID NO: 47 or SEQ ID NO: 50, optionally modified by up to 5 single amino acid changes (i.e. 1, 2, 3, 4 or 5 single amino acid substitutions, deletions and/or insertions), provided that (i) residue 32 is any amino acid, but in some embodiments is not serine (ii) residue 123 is not leucine (iii) residue 240 is not glutamate;
and (II) a second amino acid sequence selected from:
   a mutant fHbp v3 amino acid sequence, wherein: (a) the amino acid sequence has at least j % sequence identity to SEQ ID NO: 17, and/or comprises a fragment of SEQ ID NO: 17; but (b) the amino acid sequence differs from SEQ ID NO: 17 at residues L126 and E243 (and, in some embodiments, also at residue S32);
   an amino acid sequence having at least w % sequence identity to SEQ ID NO: 48, provided that (i) residue 32 is any amino acid, but in some embodiments is not serine (ii) residue 126 is not leucine (iii) residue 243 is not glutamate; or
   the amino acid sequence SEQ ID NO: 48 or SEQ ID NO: 51, optionally modified by up to 5 single amino acid changes (i.e. 1, 2, 3, 4 or 5 single amino acid substitutions, deletions and/or insertions), provided that (i) residue 32 is any amino acid, but in some embodiments is not serine (ii) residue 126 is not leucine (iii) residue 243 is not glutamate,

wherein the fusion polypeptides (a) can, when administered to a suitable mammal, elicit an antibody response that is bactericidal against both a meningococcus which expresses a v2 fHbp and a meningococcus which expresses a v3 fHbp; (b) has higher stability and lower affinity for fH than a wild-type meningococcal fHbp consisting of SEQ ID NO: 4; (c) has higher stability and lower affinity for fH than a wild-type meningococcal fHbp consisting of SEQ ID NO: 40.

The increase in stability is ideally at least 5° C. e.g. at least 10° C., 15° C., 20° C., 25° C., 30° C., 35° C. or more, as discussed above. The lower affinity is preferably at least 10-fold, and ideally at least 100-fold, as discussed above.

In one embodiment of the seventh aspect, the invention provides a polypeptide comprising a first amino acid sequence and a second amino acid sequence, wherein the first amino acid sequence is SEQ ID NO: 47 or SEQ ID NO:39 and the second amino acid sequence is SEQ ID NO: 48 or SEQ ID NO:57.

The first and second amino acid sequences can be in either order from N- to C-terminus, but it is preferred that the first sequence is upstream of the second sequence.

The first and second amino acid sequences can be joined by a linker sequence. Such linker sequence(s) will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Glyₙ where n=2, 3, 4, 5, 6, 7, 8, 9, 10 or more (SEQ ID NO: 58)), and histidine tags (i.e. Hisₙ where n=3, 4, 5, 6, 7, 8, 9, 10 or more (SEQ ID NO: 59)). Other suitable linker amino acid sequences will be apparent to those skilled in the art. One useful linker is GSGGGG (SEQ ID NO: 20), with the Gly-Ser dipeptide being formed from a BamHI restriction site, thus aiding cloning and manipulation. Another useful linker is SEQ ID NO: 21, which can optionally be preceded by a Gly-Ser dipeptide (SEQ ID NO: 22, from BamHI) or a Gly-Lys dipeptide (SEQ ID NO: 23, from HindIII).

The fusion polypeptide can also include a v1 fHbp sequence, thereby providing immune responses against all three fHbp variants with a single polypeptide i.e. the polypeptide can, when administered to a suitable mammal, elicit an antibody response that is bactericidal against a meningococcus which expresses a v1 fHbp, a meningococcus which expresses a v2 fHbp, and a meningococcus which expresses a v3 fHbp. Thus a polypeptide of the seventh aspect can also include an amino acid sequence (i) with at least i % sequence identity to SEQ ID NO: 16, and/or (ii) comprising a fragment of SEQ ID NO: 16. The value of i may be selected from 80, 85, 86, 87, 88, 89, 90, 91, 92, 93, 94, 95, 96, 97, 98, 99 or more. It is preferably 90 (i.e. the amino acid sequence has at least 90% identity to SEQ ID NO: 16) and is more preferably 95. The fragment of (ii) will generally be at least 7 amino acids long e.g. 8, 10, 12, 14, 16, 18, 20, 22, 24, 26, 28, 30, 32, 24, 26, 28, 40, 45, 50, 55, 60, 65, 70, 75, 80 or more contiguous amino acids from SEQ ID NO: 16. The fragment will typically include at least one epitope from SEQ ID NO: 16. Sharing at least 30 contiguous amino acids with SEQ ID NO: 16 will be typical, and usually a v1 fHbp amino acid sequence will include several (e.g. 2, 3, 4, 5 or more) fragments from SEQ ID NO: 16. Overall, a v1 fHbp sequence used with the seventh aspect can have at least i % sequence identity to and include several fragments of SEQ ID NO: 16.

Advantageously, the v1 fHbp sequence includes a mutation which gives it a lower affinity (as discussed above) for human fH than the same polypeptide but without the sequence differences of (b), e.g. lower affinity than a wild-type meningococcal polypeptide consisting of SEQ ID NO: 46. For instance, amino acid residue Arg-34 in SEQ ID NO: 16 (residue Arg-60 in SEQ ID NO: 1, and Arg-41 in SEQ ID NO: 46) can be mutated to Ser to disruptthe fHbp/fH interaction [19,21]. Thus a preferred v1 fHbp sequence for use with the invention comprises SEQ ID NO: 49, in which residue 34 is not arginine (e.g. SEQ ID NO: 52, where residue 34 is serine).

Where a polypeptide includes each of a v1, v2 and v3 sequence these can be present in any order from N- to C-terminus i.e. v1-v2-v3, v1-v3-v2, v2-v1-v3, v2-v3-v1, v3-v1-v2, or v3-v2-v1. The most preferred order is v2-v3-v1.

In general a preferred fHbp fusion polypeptide of the invention has an amino acid sequence of formula:

NH₂-A-[-X-L-]₃-B-COOH

wherein each X is a different variant fHbp sequence as defined herein, L is an optional linker amino acid sequence, A is an optional N-terminal amino acid sequence, and B is an optional C-terminal amino acid sequence.

The three X moieties are a v1, v2, and v3 sequence as discussed herein, so the polypeptide can, when administered to a suitable mammal, elicit an antibody response that is bactericidal against a meningococcus which expresses a v1 fHbp, a meningococcus which expresses a v2 fHbp, and a meningococcus which expresses a v3 fHbp. As mentioned above, the three variants are preferably in the order from N-terminus to C-terminus v2-v3-v1.

For each instance of [-X-L-], linker amino acid sequence -L- may be present or absent. Suitable linker sequences are discussed above.

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking. If X₁ lacks its own N-terminus methionine, -A- may provide such a methionine residue in the translated polypeptide (e.g. -A- is a single Met residue). The Met may be to the N-terminus of a linker sequence such as SEQ ID NO: 21 (i.e. SEQ ID: 24), or at the N-terminus of a short sequence (e.g. SEQ ID NO: 25).

-B- is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more (SEQ ID NO: 59)), or sequences which enhance polypeptide stability. Other suitable C-terminal amino acid sequences will be apparent to those skilled in the art. One suitable -B- moiety is SEQ ID NO: 26, in which the Leu-Glu upstream of the histidine tag arises from a XhoI restriction site.

Thus, in one embodiment, the invention provides a polypeptide comprising the amino acid sequence of SEQ ID NO: 28. From N-terminus to C-terminus, this sequence is made up from the following SEQ ID amino acid sequences:

| A | X₁ | L₁ | X₂ | L₂ | X₃ | L₃ | B |
|---|---|---|---|---|---|---|---|
| — | 50 | 20 | 51 | 20 | 52 | — | — |

By including SEQ ID NO: 24 as the N-terminal -A- moiety, the invention also provides a polypeptide comprising the amino acid sequence of SEQ ID NO: 27.

In another embodiment, the invention provides a polypeptide comprising the amino acid sequence of SEQ ID NO: 30. From N-terminus to C-terminus, this sequence is made up from the following SEQ ID amino acid sequences:

| A | X₁ | L₁ | X₂ | L₂ | X₃ | L₃ | B |
|---|---|---|---|---|---|---|---|
| — | 45 | 20 | 44 | 20 | 52 | — | — |

By including SEQ ID NO: 24 as the N-terminal -A- moiety, the invention also provides a polypeptide comprising the amino acid sequence of SEQ ID NO: 29.

### Polypeptides

Polypeptides of the invention can be prepared by various means e.g. by chemical synthesis (at least in part), by digesting longer polypeptides using proteases, by translation from RNA, by purification from cell culture (e.g. from recombinant expression or from N. meningitidis culture), etc. Heterologous expression in an E. coli host is a preferred expression route.

Polypeptides of the invention are ideally at least 100 amino acids long, e.g. 150aa, 175aa, 200aa, 225aa, or longer. They include a mutant fHbp v2 and/or v3 amino acid sequence, and the mutant fHbp v2 or v3 amino acid sequence should similarly be at least 100 amino acids long, e.g. 150aa, 175aa, 200aa, 225aa, or longer.

The fHbp is naturally a lipoprotein in N. meningitidis. It has also been found to be lipidated when expressed in E. coli with its native leader sequence or with heterologous leader sequences. Polypeptides of the invention may have a N-terminus cysteine residue, which may be lipidated e.g. comprising a palmitoyl group, usually forming tripalmitoyl-S-glyceryl-cysteine. In other embodiments the polypeptides are not lipidated.

Polypeptides are preferably prepared in substantially pure or substantially isolated form (i.e. substantially free from other Neisserial or host cell polypeptides). In general, the polypeptides are provided in a non-naturally occurring environment, e.g. they are separated from their naturally-occurring environment. In certain embodiments, the polypeptide is present in a composition that is enriched for the polypeptide as compared to a starting material. Thus purified polypeptide is provided, whereby purified means that the polypeptide is present in a composition that is substantially free of other expressed polypeptides, whereby substantially free is meant that more than 50% (e.g. ≥75%, ≥80%, ≥90%, ≥95%, or ≥99%) of total polypeptide in the composition is a polypeptide of the invention.

Polypeptides can take various forms (e.g. native, fusions, glycosylated, non-glycosylated, lipidated, disulfide bridges, etc.).

SEQ ID NOs 4, 5, 17 and 40 do not include a N-terminus methionine. If a polypeptide of the invention is produced by translation in a biological host then a start codon is required, which will provide a N-terminus methionine in most hosts. Thus a polypeptide of the invention will, at least at a nascent stage, include a methionine residue upstream of said SEQ ID NO sequence.

Cleavage of nascent sequences means that the mutant fHbp v2 or v3 amino acid sequence might itself provide the polypeptide's N-terminus. In other embodiments, however, a polypeptide of the invention can include a N-terminal sequence upstream of the mutant fHbp v2 or v3 amino acid sequence. In some embodiments the polypeptide has a single methionine at the N-terminus immediately followed by the mutant fHbp v2 or v3 amino acid sequence; in other embodiments a longer upstream sequence may be used. Such an upstream sequence may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10,9,8,7,6,5, 4, 3, 2, 1). Examples include leader sequences to direct protein trafficking, or short peptide sequences which facilitate cloning or purification (e.g. a histidine tag i.e. Hisₙ where n=4, 5, 6, 7, 8, 9, 10 or more (SEQ ID NO: 60)). Other suitable N-terminal amino acid sequences will be apparent to those skilled in the art e.g. the native upstream sequences present in SEQ ID NO: 2 or SEQ ID NO: 3.

A polypeptide of the invention may also include amino acids downstream of the final amino acid of the mutant fHbp v2 or v3 amino acid sequence. Such C-terminal extensions may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising a histidine tag i.e. Hisₙ where n=4, 5, 6, 7, 8, 9, 10 or more (SEQ ID NO: 60)), or sequences which enhance polypeptide stability. Other suitable C-terminal amino acid sequences will be apparent to those skilled in the art.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

Polypeptides can occur as single chains or associated chains.

Polypeptides of the invention may be attached or immobilised to a solid support.

Polypeptides of the invention may comprise a detectable label, e.g. a radioactive label, a fluorescent label, or a biotin label. This is particularly useful in immunoassay techniques.

As disclosed in reference 162, fHbp can be split into three domains, referred to as A, B and C. Taking SEQ ID NO: 1, the three domains are (A) 1-119, (B) 120-183 and (C) 184-274:

MNRTAFCCLSLTTALILTACSSGGGGVAADIGAGLADALTAPLDHKDKG
LQSLTLDQSVRKNEKLKLAAQGAEKTYGNGDSLNTGKLKNDKVSRFDFI
RQIEVDGQLITLESGEFQVYKQSHSALTAFQTEQIQDSEHSGKMVAKRQ
FRIGDIAGEHTSFDKLPEGGRATYRGTAFGSDDAGGKLTYTIDFAAKQG
NGKIEHLKSPELNVDLAAADIKPDGKRHAVISGSVLYNQAEKGSYSLGI
FGGKAQEVAGSAEVKTVNGIRHIGLAAKQ

The mature form of domain ‘A’, from Cys-20 at its N-terminus to Lys-119, is called ‘Amature’.

Multiple fHbp sequences are known and these can readily be aligned using standard methods. By such alignments the skilled person can identify (a) domains ‘A’ (and ‘Amature’), ‘B’ and ‘C’ in any given fHbp sequence by comparison to the coordinates in the MC58 sequence, and (b) single residues in multiple fHbp sequences e.g. for identifying substitutions. For ease of reference, however, the domains are defined below:
Domain ‘A’ in a given fHbp sequence is the fragment of that sequence which, when aligned to SEQ ID NO: 1 using a pairwise alignment algorithm, starts with the amino acid aligned to Met-1 of SEQ ID NO: 1 and ends with the amino acid aligned to Lys-119 of SEQ ID NO: 1.
Domain ‘Aₘₐₜᵤᵣₑ’ in a given fHbp sequence is the fragment of that sequence which, when aligned to SEQ ID NO: 1 using a pairwise alignment algorithm, starts with the amino acid aligned to Cys-20 of SEQ ID NO: 1 and ends with the amino acid aligned to Lys-119 of SEQ ID NO: 1.
Domain ‘B’ in a given fHbp sequence is the fragment of that sequence which, when aligned to SEQ ID NO: 1 using a pairwise alignment algorithm, starts with the amino acid aligned to Gln-120 of SEQ ID NO: 1 and ends with the amino acid aligned to Gly-183 of SEQ ID NO: 1.
Domain ‘C’ in a given fHbp sequence is the fragment of that sequence which, when aligned to SEQ ID NO: 1 using a pairwise alignment algorithm, starts with the amino acid aligned to Lys-184 of SEQ ID NO: 1 and ends with the amino acid aligned to Gln-274 of SEQ ID NO: 1.

The preferred pairwise alignment algorithm for defining the domains is the Needleman-Wunsch global alignment algorithm [156], using default parameters (e.g. with Gap opening penalty=10.0, and with Gap extension penalty=0.5, using the EBLOSUM62 scoring matrix). This algorithm is conveniently implemented in the needle tool in the EMBOSS package [157].

In some embodiments, a mutant fHbp v2 or v3 amino acid sequence of the invention is truncated to remove its domain A. In general, however, it is preferred that the mutant flbp v2 or v3 amino acid sequence should include both a N-terminal β-barrel and a C-terminal β-barrel.

In some embodiments, a polypeptide comprises an amino acid sequence as described above, except that up to 10 amino acids (i.e. 1, 2, 3, 4, 5, 6, 7, 8, 9 or 10) at the N-terminus and/or up to 10 amino acids (i.e. 1, 2, 3, 4, 5, 6, 7, 8, 9 or 10) at the C-terminus are deleted.

### Nucleic Acids

The invention provides nucleic acids encoding a polypeptide of the invention as defined above.

Nucleic acids of the invention may be prepared in many ways, e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acids of the invention can take various forms e.g. single-stranded, double-stranded, vectors, primers, probes, labelled, unlabelled, etc.

Nucleic acids of the invention are preferably in isolated or substantially isolated form.

The term “nucleic acid” includes DNA and RNA, and also their analogues, such as those containing modified backbones, and also peptide nucleic acids (PNA), etc.

Nucleic acid according to the invention may be labelled e.g. with a radioactive or fluorescent label.

The invention also provides vectors (such as plasmids) comprising nucleotide sequences of the invention (e.g. cloning or expression vectors, such as those suitable for nucleic acid immunisation) and host cells transformed with such vectors.

### Bactericidal Responses

Preferred polypeptides of the invention can elicit antibody responses that are bactericidal against meningococci. Bactericidal antibody responses are conveniently measured in mice and are a standard indicator of vaccine efficacy (e.g. see end-note 14 of ref. 36; also ref. 37). Thus the antibodies will be bactericidal against a test strain in a suitable serum bactericidal assay (SBA).

Polypeptides of the first aspect of the invention can preferably elicit an antibody response, e.g., in a mouse, which is bactericidal against a N. meningitidis strain which expresses a v2 fHbp sequence e.g. one or more of strains 961-5945, 2996, 96217, 312294, 11327, a22, gb013 (=MO1-240013), e32, m1090, m4287, 860800, 599, 95N477, 90-18311, ell, m986, m2671, 1000, m1096, m3279, bz232, dk353, m3697, ngh38, and/or L93/4286. Bactericidal responses can for instance be assessed against var2 strain M2091 (ATCC 13091).

Preferred polypeptides of the first aspect of the invention can elicit antibodies in a mouse which are bactericidal against strain M2091 in a serum bactericidal assay.

Polypeptides of the second aspect of the invention can preferably elicit an antibody response, e.g., in a mouse, which is bactericidal against a N. meningitidis strain which expresses a v3 fHbp sequence e.g. one or more of strains M1239, 16889, gb355 (=MO1-240355), m3369, m3813, ngp165. Bactericidal responses can for instance be assessed against var3 strain MO1-240355, which is a Neisseria MLST reference strains (id 19265 in ref. 38) which has been fully sequenced (see EMBL ID CP002422 [39]) Preferred polypeptides of the second aspect of the invention can elicit antibodies in a mouse which are bactericidal against strain MO1-240355 in a serum bactericidal assay.

For example, an immunogenic composition comprising these polypeptides can provide a serum bactericidal titer of ≥1:4 using the Goldschneider assay with human complement [40-42], and/or providing a serum bactericidal titer of ≥1:128 using baby rabbit complement.

### Immunisation

Polypeptides of the invention may be used as the active ingredient of immunogenic compositions, and so the invention provides an immunogenic composition (e.g. a vaccine) comprising a polypeptide of the invention.

The invention also provides a method for raising an antibody response in a mammal, e.g, a mouse or a human, comprising administering an immunogenic composition of the invention to the mammal. The antibody response is preferably a protective and/or bactericidal antibody response. The invention also provides polypeptides of the invention for use in such methods.

The invention also provides a method for protecting a mammal, e.g., a mouse or a human, against a Neisserial (e.g. meningococcal) infection, comprising administering to the mammal an immunogenic composition of the invention.

The invention provides polypeptides of the invention for use as medicaments (e.g. as immunogenic compositions or as vaccines) or as diagnostic reagents. It also provides the use of nucleic acid or polypeptide of the invention in the manufacture of a medicament for preventing Neisserial (e.g. meningococcal) infection in a mammal, e.g., a mouse or a human.

The mammal is preferably a human. The human may be an adult or, preferably, a child. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia. For instance, they are suitable for active immunisation of individuals against invasive meningococcal disease caused by N. meningitidis (for example in serogroup B).

Efficacy of therapeutic treatment can be tested by monitoring Neisserial infection after administration of the composition of the invention. Efficacy of prophylactic treatment can be tested by monitoring immune responses against fHbp after administration of the composition. Immunogenicity of compositions of the invention can be determined by administering them to test subjects (e.g. children 12-16 months age, or animal models) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

Preferred compositions of the invention can confer an antibody titre in a human patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

The invention may be used to elicit systemic and/or mucosal immunity.

Compositions of the invention will generally be administered directly to a human patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is about 0.5 ml (e.g. as seen in the BEXSERO (vaccine) product).

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined. For instance, the BEXSERO (vaccine) product is administered as two or three doses given not less than 1 month or not less than 2 months apart, depending on the subject (e.g. infants or others).

The immunogenic composition of the invention will generally include a pharmaceutically acceptable carrier, which can be any substance that does not itself induce the production of antibodies harmful to the patient receiving the composition, and which can be administered without undue toxicity. Pharmaceutically acceptable carriers can include liquids such as water, saline, glycerol and ethanol. Auxiliary substances, such as wetting or emulsifying agents, pH buffering substances, and the like, can also be present in such vehicles. A thorough discussion of suitable carriers is available in ref. 43. For example, the BEXSERO (vaccine) product includes sodium chloride, histidine, sucrose, aluminium hydroxide, and water for injections.

Neisserial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared. Compositions suitable for parenteral injection (e.g. into muscle) are most preferred.

The composition is preferably sterile. It is preferably pyrogen-free. It is preferably buffered e.g. at between pH 6 and pH 8, generally around pH 7. Where a composition comprises an aluminium hydroxide salt, it is preferred to use a histidine buffer [44]. Compositions of the invention may be isotonic with respect to humans.

Immunogenic compositions comprise an immunologically effective amount of immunogen, as well as any other of other specified components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The composition may be administered in conjunction with other immunoregulatory agents.

Adjuvants which may be used in compositions of the invention include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolones, or mixtures thereof. Other substances that act as immunostimulating agents are disclosed in chapter 7 of ref. 45.

The use of an aluminium hydroxide and/or aluminium phosphate adjuvant is particularly preferred, and polypeptides are generally adsorbed to these salts. These salts include oxyhydroxides and hydroxyphosphates (e.g. see chapters 8 & 9 of ref. 45). The salts can take any suitable form (e.g. gel, crystalline, amorphous, etc.). A1⁺⁺⁺ should be present at <1 mg/dose.

The most preferred adjuvant is aluminium hydroxide, as used in the BEXSERO (vaccine) product. Polypeptides in a composition of the invention can be adsorbed to this adjuvant, as seen in the BEXSERO (vaccine) product. It can be included at about 1 mg/ml A1⁺⁺⁺ (i.e. 0.5 mg per 0.5 ml dose)

### Further Antigenic Components

Compositions of the invention include mutant v2 and/or v3 fHbp sequence. It is useful if the composition should not include complex or undefined mixtures of antigens e.g. it is preferred not to include outer membrane vesicles in the composition. Polypeptides of the invention are preferably expressed recombinantly in a heterologous host and then purified.

As well as including a fHbp polypeptide, a composition of the invention may also include one or more further neisserial immunogen(s), as a vaccine which targets more than one immunogen per bacterium decreases the possibility of selecting escape mutants. Thus a composition can include a second polypeptide that, when administered to a suitable mammal, elicits an antibody response that is bactericidal against meningococcus. The second polypeptide can be a meningococcal fHbp, but will often not be a fHbp e.g. it may be a NHBA sequence, a NadA sequence, etc.

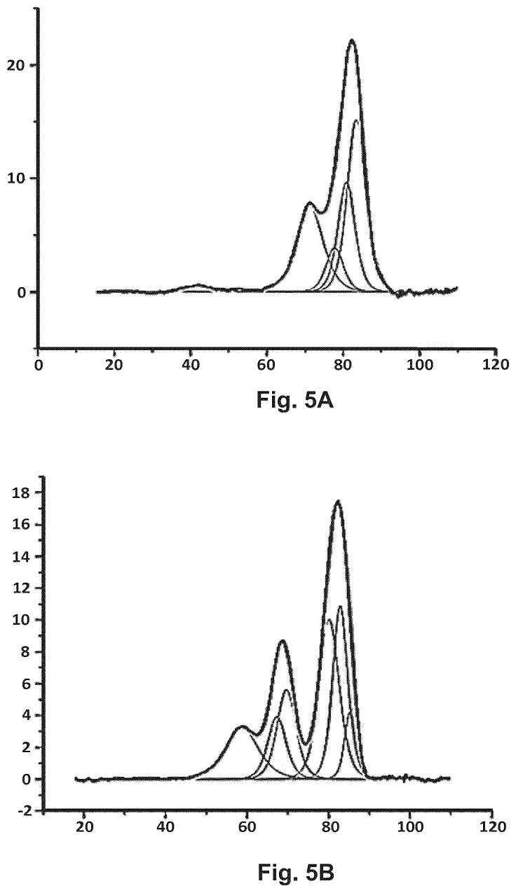

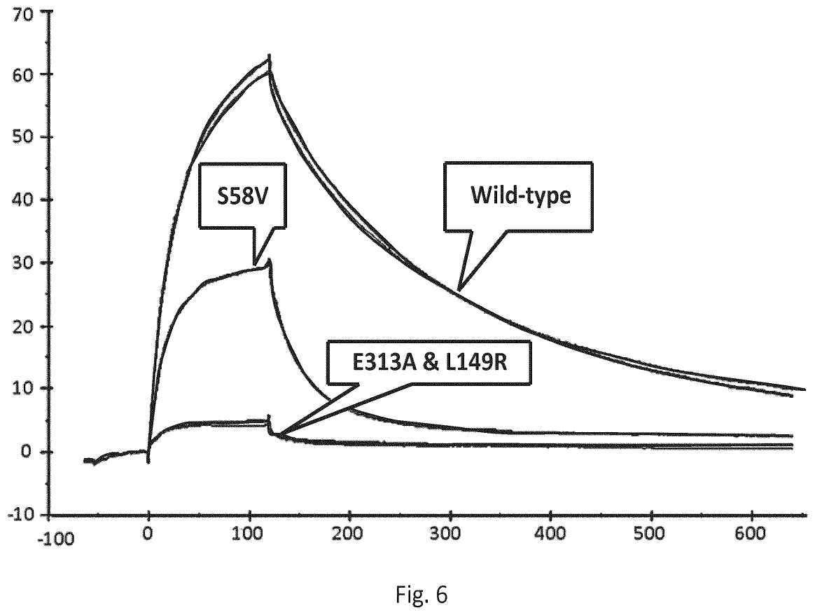

A composition of the invention may include a NHBA antigen. The NHBA antigen was included in the published genome sequence for meningococcal serogroup B strain MC58 [46] as gene NMB2132 (GenBank accession number GI:7227388; SEQ ID NO: 6 herein). The sequences of NHBA antigen from many strains have been published since then. For example, allelic forms of NHBA can be seen in FIGS. 5 and 15 of reference 47, and in example 13 and FIG. 21 of reference 1 (SEQ IDs 3179 to 3184 therein). Various immunogenic fragments of the NHBA antigen have also been reported. Preferred 287 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) comprising a fragment of at least ‘n’ consecutive amino acids of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 6. The most useful NHBA antigens of the invention can elicit antibodies which, after administration to a subject, can bind to a meningococcal polypeptide consisting of amino acid sequence SEQ ID NO: 6. Advantageous NHBA antigens for use with the invention can elicit bactericidal anti-meningococcal antibodies after administration to a subject.

A composition of the invention may include a NadA antigen. The NadA antigen was included in the published genome sequence for meningococcal serogroup B strain MC58 [46] as gene NMB1994 (GenBank accession number GI:7227256; SEQ ID NO: 7 herein). The sequences of NadA antigen from many strains have been published since then, and the protein's activity as a Neisserial adhesin has been well documented. Various immunogenic fragments of NadA have also been reported.

Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) comprising a fragment of at least ‘n’ consecutive amino acids of SEQ ID NO: 7, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 7. The most useful NadA antigens of the invention can elicit antibodies which, after administration to a subject, can bind to a meningococcal polypeptide consisting of amino acid sequence SEQ ID NO: 7. Advantageous NadA antigens for use with the invention can elicit bactericidal anti-meningococcal antibodies after administration to a subject. SEQ ID NO: 15 is one such fragment.

A composition of the invention may include a NspA antigen. The NspA antigen was included in the published genome sequence for meningococcal serogroup B strain MC58 [46] as gene NMB0663 (GenBank accession number GI:7225888; SEQ ID NO: 8 herein). The antigen was previously known from references 48 & 49. The sequences of NspA antigen from many strains have been published since then. Various immunogenic fragments of NspA have also been reported. Preferred NspA antigens for use with the invention comprise n amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 8. The most useful NspA antigens of the invention can elicit antibodies which, after administration to a subject, can bind to a meningococcal polypeptide consisting of amino acid sequence SEQ ID NO: 8. Advantageous NspA antigens for use with the invention can elicit bactericidal anti-meningococcal antibodies after administration to a subject.

Compositions of the invention may include a meningococcal HmbR antigen. The full-length HmbR sequence was included in the published genome sequence for meningococcal serogroup B strain MC58 [46] as gene NMB1668 (SEQ ID NO: 9 herein). The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i % sequence identity to SEQ ID NO: 9, where the value of i is 50, 60, 70, 80, 90, 95, 99 or more. In other embodiments a HmbR sequence used according to the invention may comprise a fragment of at least j consecutive amino acids from SEQ ID NO: 9, where the value ofj is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i % sequence identity to SEQ ID NO: 9 and/or (ii) comprising a fragment of at least j consecutive amino acids from SEQ ID NO: 9. Preferred fragments of j amino acids comprise an epitope from SEQ ID NO: 9. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 50. The most useful HmbR antigens of the invention can elicit antibodies which, after administration to a subject, can bind to a meningococcal polypeptide consisting of amino acid sequence SEQ ID NO: 9. Advantageous HmbR antigens for use with the invention can elicit bactericidal anti-meningococcal antibodies after administration to a subject.

A composition of the invention may include a NhhA antigen. The NhhA antigen was included in the published genome sequence for meningococcal serogroup B strain MC58 [46] as gene NMB0992 (GenBank accession number GI:7226232; SEQ ID NO: 10 herein). The sequences of NhhA antigen from many strains have been published since e.g. refs 47 & 51, and various immunogenic fragments of NhhA have been reported. It is also known as Hsf. Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 10. The most useful NhhA antigens of the invention can elicit antibodies which, after administration to a subject, can bind to a meningococcal polypeptide consisting of amino acid sequence SEQ ID NO: 10. Advantageous NhhA antigens for use with the invention can elicit bactericidal anti-meningococcal antibodies after administration to a subject.

A composition of the invention may include an App antigen. The App antigen was included in the published genome sequence for meningococcal serogroup B strain MC58 [46] as gene NMB1985 (GenBank accession number GI:7227246; SEQ ID NO: 11 herein). The sequences of App antigen from many strains have been published since then. Various immunogenic fragments of App have also been reported. Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 11. The most useful App antigens of the invention can elicit antibodies which, after administration to a subject, can bind to a meningococcal polypeptide consisting of amino acid sequence SEQ ID NO: 11. Advantageous App antigens for use with the invention can elicit bactericidal anti-meningococcal antibodies after administration to a subject.

A composition of the invention may include an Omp85 antigen. The Omp85 antigen was included in the published genome sequence for meningococcal serogroup B strain MC58 [46] as gene NMB0182 (GenBank accession number GI:7225401; SEQ ID NO: 12 herein). The sequences of Omp85 antigen from many strains have been published since then. Further information on Omp85 can be found in references 52 and 53. Various immunogenic fragments of Omp85 have also been reported. Preferred Omp85 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 12. The most useful Omp85 antigens of the invention can elicit antibodies which, after administration to a subject, can bind to a meningococcal polypeptide consisting of amino acid sequence SEQ ID NO: 12. Advantageous Omp85 antigens for use with the invention can elicit bactericidal anti-meningococcal antibodies after administration to a subject.

A composition of the invention may include a 936 antigen. The 936 antigen was included in the published genome sequence for meningococcal serogroup B strain MC58 [46] as gene NMB2091 (SEQ ID NO: 13 herein). Preferred 936 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) comprising a fragment of at least ‘n’ consecutive amino acids of SEQ ID NO: 13, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 13. The most useful 936 antigens of the invention can elicit antibodies which, after administration to a subject, can bind to a meningococcal polypeptide consisting of amino acid sequence SEQ ID NO: 13. The 936 antigen is a good fusion partner for fHbp (e.g. see references 54 & 55).

A composition may comprise: a polypeptide comprising SEQ ID NO: 14; a polypeptide comprising SEQ ID NO: 15; and a polypeptide of the invention comprising a mutant fHbp v2 amino acid sequence and SEQ ID NO: 13 (cf refs. 54 & 55).

A composition may comprise: a polypeptide comprising SEQ ID NO: 14; a polypeptide comprising SEQ ID NO: 15; and a polypeptide of the invention comprising a mutant fHbp v3 amino acid sequence and SEQ ID NO: 13 (cf refs. 54 & 55).

In some embodiments, a polypeptide of the invention is combined with a further meningococcal fHbp sequence. In particular, a v2 polypeptide can be combined with a v1 and/or a v3 polypeptide to increase the spectrum of strain coverage [160]. Thus a composition can comprise: (i) a polypeptide of the invention comprising a mutant fHbp v2 amino acid sequence; and (ii) a v1 fHbp polypeptide and/or a v3 fHbp polypeptide. In other embodiments, a polypeptide of the invention can comprise (i) a mutant fHbp v2 amino acid sequence and (ii) a v1 fHbp amino acid sequence and/or a v3 fHbp amino acid sequence. Thus the v1 and/or v3 sequences can be combined with the mutant v2 sequence as separate entities in a composition (or within a fusion polypeptide, as discussed above).

Similarly, a v3 polypeptide can be combined with a v1 and/or a v2 polypeptide to increase the spectrum of strain coverage [160]. Thus a composition can comprise: (i) a polypeptide of the invention comprising a mutant fHbp v3 amino acid sequence; and (ii) a v1 fHbp polypeptide and/or a v2 fHbp polypeptide. In other embodiments, a polypeptide of the invention can comprise (i) a mutant fHbp v3 amino acid sequence and (ii) a v1 fHbp amino acid sequence and/or a v2 fHbp amino acid sequence. Thus the v1 and/or v2 sequences can be combined with the mutant v3 sequence as separate entities in a composition (or within a fusion polypeptide, as discussed above).

Moreover, mutant v2 and v3 polypeptides can be combined with each other to increase strain coverage. Thus a composition can comprise: (i) a polypeptide of the invention comprising a mutant fHbp v2 amino acid sequence; (ii) a polypeptide of the invention comprising a mutant fHbp v3 amino acid sequence; and (iii) a fHbp v1 polypeptide. In other embodiments, a polypeptide of the invention can comprise (i) a mutant fHbp v2 amino acid sequence (ii) a mutant v3 fHbp amino acid sequence and (iii) a fHbp v1 amino acid sequence. Thus the mutant v2 and v3 sequences can be combined with a v1 sequence as separate entities in a composition (or within a fusion polypeptide, as discussed above).

The v1 sequence can be a wild-type sequence or a mutant sequence. A v1 fHbp can comprise (a) an amino acid sequence which has at least k % identity to SEQ ID NO: 16, and/or (b) a fragment of SEQ ID NO: 16. Information about ‘k’ and fragments are given above. The fragment will typically include at least one epitope from SEQ ID NO: 16, and the v1 fHbp polypeptide will include at least one epitope which is not present in the v2 or v3 amino acid sequence of the invention, such that antibodies elicited by the v1 fHbp can recognise v1 strains. Ideally, the v1 fHbp can elicit antibodies which are bactericidal against v1 strains e.g. against strain MC58 (available from the ATCC as ‘BAA-335’). The v1 fHbp can include an amino acid mutation which disrupts its ability to bind to fH.

A v2 fHbp can comprise (a) an amino acid sequence which has at least k % identity to SEQ ID NO: 5, and/or (b) a fragment of SEQ ID NO: 5. Information about ‘k’ and fragments are given above. The fragment will typically include at least one epitope from SEQ ID NO: 5, and the v2 fHbp polypeptide will include at least one epitope which is not present in the v3 amino acid sequence of the invention, such that antibodies elicited by the v2 fHbp can recognise v2 strains. Ideally, the v2 fHbp can elicit antibodies which are bactericidal against v2 strains e.g. against strain M2091 (ATCC 13091). The v2 fHbp can be a polypeptide of the first aspect of the invention.

A v3 fHbp can comprise (a) an amino acid sequence which has at least k % identity to SEQ ID NO: 17, and/or (b) a fragment of SEQ ID NO: 17. Information about ‘k’ and fragments are given above. The fragment will typically include at least one epitope from SEQ ID NO: 17, and the v3 fHbp polypeptide will include at least one epitope which is not present in the v2 amino acid sequence of the invention, such that antibodies elicited by the v3 fHbp can recognise v3 strains. Ideally, the v3 fHbp can elicit antibodies which are bactericidal against v3 strains e.g. against strain M01-240355. The v3 fHbp can be a polypeptide of the second aspect of the invention.

In addition to Neisserial polypeptide antigens, the composition may include antigens for immunising against other diseases or infections. For example, the composition may include one or more of the following further antigens:
a saccharide antigen from N. meningitidis serogroup A, C, W135 and/or Y, such as the saccharide disclosed in ref. 56 from serogroup C (see also ref. 57) or in ref. 58.
a saccharide antigen from Streptococcus pneumoniae [e.g. 59, 60, 61].
an antigen from hepatitis A virus, such as inactivated virus [e.g. 62, 63].
an antigen from hepatitis B virus, such as the surface and/or core antigens [e.g. 63, 64].
a diphtheria antigen, such as a diphtheria toxoid [e.g. chapter 3 of ref. 65] e.g. the CRM197 mutant [e.g. 66].
a tetanus antigen, such as a tetanus toxoid (e.g. chapter 4 of ref. 65).
an antigen from Bordetella pertussis, such as pertussis holotoxin (PT) and filamentous haemagglutinin (FHA) from B. pertussis, optionally also in combination with pertactin and/or agglutinogens 2 and 3 (e.g. refs. 67 & 68).
a saccharide antigen from Haemophilus influenzae B [e.g. 57].
polio antigen(s) [e.g. 69, 70] such as IPV.
measles, mumps and/or rubella antigens (e.g. chapters 9, 10 & 11 of ref. 65).
influenza antigen(s) (e.g. chapter 19 of ref. 65), such as the haemagglutinin and/or neuraminidase surface proteins.
an antigen from Moraxella catarrhalis [e.g. 71].
an protein antigen from Streptococcus agalactiae (group B streptococcus) [e.g. 72, 73].
a saccharide antigen from Streptococcus agalactiae (group B streptococcus).
an antigen from Streptococcus pyogenes (group A streptococcus) [e.g. 73, 74, 75].
an antigen from Staphylococcus aureus [e.g. 76].

The composition may comprise one or more of these further antigens.

Toxic protein antigens may be detoxified where necessary (e.g. detoxification of pertussis toxin by chemical and/or genetic means [68]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates are discussed in more detail below.

Antigens in the composition will typically be present at a concentration of at least lpg/ml each. In general, the concentration of any given antigen will be sufficient to elicit an immune response against that antigen.

Immunogenic compositions of the invention may be used therapeutically (i.e. to treat an existing infection) or prophylactically (i.e. to prevent future infection).

As an alternative to using proteins antigens in the immunogenic compositions of the invention, nucleic acid (which could be RNA, such as a self-replicating RNA, or DNA, such as a plasmid) encoding the antigen may be used.

In some embodiments a composition of the invention comprises in addition to the fHbp sequence, conjugated capsular saccharide antigens from 1, 2, 3 or 4 of meningococcus serogroups A, C, W135 and Y. In other embodiments a composition of the invention comprises in addition to the fHbp sequence, at least one conjugated pneumococcal capsular saccharide antigen.

### Meningococcus Serogroups Y, W135, C and A

Current serogroup C vaccines (MENJUGATE (vaccine) [56,77], MENINGITEC (vaccine) andNEISVAC-C(vaccine)) include conjugated saccharides. MENJUGATE (vaccine) and MENINGITEC (vaccine) have oligosaccharide antigens conjugated to a CRM197 carrier, whereas NEISVAC-C(vaccine) uses the complete polysaccharide (de-O-acetylated) conjugated to a tetanus toxoid carrier. The MENACTRA (vaccine) vaccine contains conjugated capsular saccharide antigens from each of serogroups Y, W135, C and A.

Compositions of the present invention may include capsular saccharide antigens from one or more of meningococcus serogroups Y, W135, C and A, wherein the antigens are conjugated to carrier protein(s) and/or are oligosaccharides. For example, the composition may include a capsular saccharide antigen from: serogroup C; serogroups A and C; serogroups A, C and W135; serogroups A, C and Y; serogroups C, W135 and Y; or from all four of serogroups A, C, W135 and Y.

A typical quantity of each meningococcal saccharide antigen per dose is between 1 μg and 20 μg e.g. about 1 μg, about 2.5 μg, about 4 μg, about 5 μg, or about 10 μg (expressed as saccharide).

Where a mixture comprises capsular saccharides from both serogroups A and C, the ratio (w/w) of MenA saccharide:MenC saccharide may be greater than 1 (e.g. 2:1, 3:1, 4:1, 5:1, 10:1 or higher).

Where a mixture comprises capsular saccharides from serogroup Y and one or both of serogroups C and W135, the ratio (w/w) of MenY saccharide:MenW135 saccharide may be greater than 1 (e.g. 2:1, 3:1, 4:1, 5:1, 10:1 or higher) and/or that the ratio (w/w) of MenY saccharide:MenC saccharide may be less than 1 (e.g. 1:2, 1:3, 1:4, 1:5, or lower). Preferred ratios (w/w) for saccharides from serogroups A:C:W135:Y are: 1:1:1:1; 1:1:1:2; 2:1:1:1; 4:2:1:1; 8:4:2:1; 4:2:1:2; 8:4:1:2; 4:2:2:1; 2:2:1:1; 4:4:2:1; 2:2:1:2; 4:4:1:2; and 2:2:2:1. Preferred ratios (w/w) for saccharides from serogroups C:W135:Y are: 1:1:1; 1:1:2; 1:1:1; 2:1:1; 4:2:1; 2:1:2; 4:1:2; 2:2:1; and 2:1:1. Using a substantially equal mass of each saccharide is preferred.

Capsular saccharides may be used in the form of oligosaccharides. These are conveniently formed by fragmentation of purified capsular polysaccharide (e.g. by hydrolysis), which will usually be followed by purification of the fragments of the desired size.

Fragmentation of polysaccharides is preferably performed to give a final average degree of polymerisation (DP) in the oligosaccharide of less than 30 (e.g. between 10 and 20, preferably around 10 for serogroup A; between 15 and 25 for serogroups W135 and Y, preferably around 15-20; between 12 and 22 for serogroup C; etc.). DP can conveniently be measured by ion exchange chromatography or by colorimetric assays [78].

If hydrolysis is performed, the hydrolysate will generally be sized in order to remove short-length oligosaccharides [57]. This can be achieved in various ways, such as ultrafiltration followed by ion-exchange chromatography. Oligosaccharides with a degree of polymerisation of less than or equal to about 6 are preferably removed for serogroup A, and those less than around 4 are preferably removed for serogroups W135 and Y.

Preferred MenC saccharide antigens are disclosed in reference 77, as used inMENJUGATE (vaccine).

The saccharide antigen may be chemically modified. This is particularly useful for reducing hydrolysis for serogroup A [79]. De-O-acetylation of meningococcal saccharides can be performed. For oligosaccharides, modification may take place before or after depolymerisation.

Where a composition of the invention includes a MenA saccharide antigen, the antigen is preferably a modified saccharide in which one or more of the hydroxyl groups on the native saccharide has/have been replaced by a blocking group [79]. This modification improves resistance to hydrolysis.

### Covalent Conjugation

Capsular saccharides in compositions of the invention will usually be conjugated to carrier protein(s).

In general, conjugation enhances the immunogenicity of saccharides as it converts them from T-independent antigens to T-dependent antigens, thus allowing priming for immunological memory. Conjugation is particularly useful for paediatric vaccines and is a well known technique.

Typical carrier proteins are bacterial toxins, such as diphtheria or tetanus toxins, or toxoids or mutants thereof. The CRM₁₉₇ diphtheria toxin mutant [80] is useful, and is the carrier in the PREVNAR (vaccine) product. Other suitable carrier proteins include the N. meningitidis outer membrane protein complex [81], synthetic peptides [82,83], heat shock proteins [84,85], pertussis proteins [86,87], cytokines [88], lymphokines [88], hormones [88], growth factors [88], artificial proteins comprising multiple human CD4⁺ T cell epitopes from various pathogen-derived antigens [89] such as N19 [90], protein D from H. influenzae [91-93], pneumolysin [94] or its non-toxic derivatives [95], pneumococcal surface protein PspA [96], iron-uptake proteins [97], toxin A or B from C. difficile [98], recombinant P. aeruginosa exoprotein A (rEPA) [99], etc.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [100,101,etc.]). Other suitable techniques use carbodiimides, hydrazides, active esters, norborane, p-nitrobenzoic acid, N-hydroxysuccinimide, S-NHS, EDC, TSTU, etc.

Linkages via a linker group may be made using any known procedure, for example, the procedures described in references 102 and 103. One type of linkage involves reductive amination of the polysaccharide, coupling the resulting amino group with one end of an adipic acid linker group, and then coupling a protein to the other end of the adipic acid linker group [104,105]. Other linkers include B-propionamido [106], nitrophenyl-ethylamine [107], haloacyl halides [108], glycosidic linkages [109], 6-aminocaproic acid [110], ADH [111], C₄ to C12 moieties [112] etc. As an alternative to using a linker, direct linkage can be used. Direct linkages to the protein may comprise oxidation of the polysaccharide followed by reductive amination with the protein, as described in, for example, references 113 and 114.

A process involving the introduction of amino groups into the saccharide (e.g. by replacing terminal =0 groups with —NH₂) followed by derivatisation with an adipic diester (e.g. adipic acid N-hydroxysuccinimido diester) and reaction with carrier protein is preferred. Another preferred reaction uses CDAP activation with a protein D carrier e.g. for MenA or MenC.

### Outer Membrane Vesicles (OMVs)

It is preferred that compositions of the invention should not include complex or undefined mixtures of antigens, which are typical characteristics of OMVs. However, the invention can be used in conjunction with OMVs, as fHbp has been found to enhance their efficacy [4], whether by simple mixing or by over-expressing the polypeptides of the invention in the strains used for OMV preparation.

This approach may be used in general to improve preparations of N. meningitidis serogroup B microvesicles [115], ‘native OMVs’ [116], blebs or outer membrane vesicles (e.g. refs. 117 to 123, etc.).

Typical outer membrane vesicles are prepared artificially from bacteria, and may be prepared using detergent treatment (e.g. with deoxycholate), or by non-detergent means (e.g. see reference 127). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [124 & 125] being preferred for treatingNeisseria) at a pH sufficiently high not to precipitate the detergent [126]. Other techniques may be performed substantially in the absence of detergent [127,128] using techniques such as sonication, homogenisation, microfluidisation, cavitation, osmotic shock, grinding, French press, blending, etc. Methods using no or low detergent can retain useful antigens such as NspA and fHbp [127]. Thus OMVs used with the invention may be prepared using an OMV extraction buffer having about 0.5% deoxycholate or lower e.g. about 0.2%, about 0.1%, <0.05% or even zero.

The vesicles known as MVs (membrane vesicles) and NOMVs (native outer membrane vesicles) are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 135 & 136 describe Neisseria with high MV production.

Vesicles may be prepared from bacteria which have been genetically manipulated [129-132] e.g. to increase immunogenicity (e.g. hyper-express immunogens), to reduce toxicity, to inhibit capsular polysaccharide synthesis, to down-regulate PorA expression, etc. They may be prepared from hyperblebbing strains [133-136]. Vesicles from bacteria with different class I outer membrane protein subtypes may be used e.g. six different subtypes [137,138] using two different genetically-engineered vesicle populations each displaying three subtypes, or nine different subtypes using three different genetically-engineered vesicle populations each displaying three subtypes, etc. Useful subtypes include: P1.7,16; P1.5-1,2-2; P1.19,15-1; P1.5-2,10; P1.12-1,13; P1.7-2,4; P1.22,14; P1.7-1,1; P1.18-1,3,6. In general, however, it is preferred for the present invention to prepare OMVs from a wild-type meningococcus strain.

Vesicles for use with the invention can thus be prepared from any wild-type meningococcal strain. The vesicles will usually be from a serogroup B strain, but it is possible to prepare them from serogroups other than B (e.g. reference discloses 126 a process for serogroup A), such as A, C, W135 or Y. The strain may be of any serotype (e.g. 1, 2a, 2b, 4, 14, 15, 16, etc.), any serosubtype (e.g. P1.4), and any immunotype (e.g. L1; L2; L3; L3,7; L3,7,9; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. Most preferably, OMVs are prepared from the strain NZ98/254, or another strain with the P1.4 PorA serosubtype. The invention advantageously uses the same OMVs which are used in the BEXSERO (vaccine) and MENZB (vaccine) products, prepared from the strain NZ98/254.

Vesicles will generally include meningococcal lipooligosaccharides (LOS, also known as LPS, lipopolysaccharide), but the pyrogenic effect of LOS in OMVs is much lower than seen with the same amount of purified LOS, and adsorption of OMVs to aluminium hydroxide further reduces pyrogenicity. LOS levels are expressed in International Units (IU) of endotoxin and can be tested by the LAL assay (limulus amebocyte lysate). Preferably, LOS is present at less than 2000 IU per g of OMV protein.

When LOS is present in a vesicle it is possible to treat the vesicle so as to link its LOS and protein components (“intra-bleb” conjugation [139]).

A useful process for OMV purification is described in reference 140 and involves ultrafiltration on crude OMVs, rather than instead of high speed centrifugation. The process may involve a step of ultracentrifugation after the ultrafiltration takes place. The process may involve a step of ultracentrifugation after the ultrafiltration takes place. OMVs can also be purified using the two stage size filtration process described in ref. 152.

OMVs can usefully be suspended in a sucrose solution after they have been prepared.

### Host Cells

The invention provides a bacterium which expresses a polypeptide of the invention. The bacterium may be a meningococcus or an E. coli. The bacterium may constitutively express the polypeptide, but in some embodiments expression may be under the control of an inducible promoter. The bacterium may hyper-express the polypeptide (cf ref. 141). Expression of the polypeptide is ideally not phase variable.

The invention also provides outer membrane vesicles prepared from a bacterium of the invention (particularly from a meningococcus). It also provides aprocess for producing vesicles from a bacterium of the invention. Vesicles prepared from these strains preferably include the polypeptide of the invention, which should be in an immunoaccessible form in the vesicles i.e. an antibody which can bind to purified polypeptide of the invention should also be able to bind to the polypeptide which is present in the vesicles.

Bacteria of the invention may, in addition to encoding a polypeptide of the invention, have one or more further modifications. For instance, they may have a modified fur gene [142]. Expression of nspA expression may be up-regulated with concomitant porA and cps knockout. Further knockout mutants ofN.meningitidis for OMV production are disclosed e.g. in reference 139. Reference 143 discloses the construction of vesicles from strains modified to express six different PorA subtypes. Mutant Neisseria with low endotoxin levels, achieved by knockout of enzymes involved in LPS biosynthesis, may also be used [144,145].MutantNeisseria engineered to reduce or switch off expression of at least one gene involved in rendering toxic the lipid A portion of LPS, in particular of lpxl1 gene, can be used with the invention [146]. Similarly, mutant Neisseria engineered to reduce or switch off expression of at least one gene involved in the capsular polysaccharide synthesis or export, in particular of synX and/or ctrA genes can be used with the invention. These or others mutants can all be used with the invention.

In some embodiments a strain may have been down-regulated for PorA expression e.g. in which the amount of PorA has been reduced by at least 20% (e.g. ≥30%, ≥40%, ≥50%, ≥60%, ≥70%, ≥80%, ≥90%, ≥95%, etc.), or even knocked out, relative to wild-type levels (e.g. relative to strain H44/76). In some embodiments a strain may hyper-express (relative to the corresponding wild-type strain) certain proteins. For instance, strains may hyper-express NspA, protein 287 [117], fHbp [141](including fHbp of the invention), TbpA and/or TbpB [147], Cu,Zn-superoxide dismutase, HmbR, etc.

A gene encoding a polypeptide of the invention may be integrated into the bacterial chromosome or may be present in episomal form e.g. within a plasmid.

Advantageously for vesicle production, a meningococcus may be genetically engineered to ensure that expression of the polypeptide is not subject to phase variation. Methods for reducing or eliminating phase variability of gene expression in meningococcus are disclosed in reference 148. For example, a gene may be placed under the control of a constitutive or inducible promoter, or by removing or replacing the DNA motif which is responsible for its phase variability.

In some embodiments a strain may include one or more of the knockout and/or hyper-expression mutations disclosed in references 122, 129, 133, and 139. For instance, following the guidance and nomenclature in these four documents, useful genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB; or (d) CtrA, CtrB, CtrD, FrpB, OpA, OpC, PilC, PorB, SiaD, SynA, SynB, SynX and/or SynC.

Where a mutant strain is used, in some embodiments it may have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no a chain.

Depending on the meningococcal strain used for preparing the vesicles, they may or may not include the strain's native fHbp antigen [149].

In one preferred embodiment, a meningococcus does not express a functional MltA protein. As discussed in refs. 150 & 151, knockout of MltA (the membrane-bound lytic transglycosylase, also known as GNA33) in meningococcus provides bacteria which spontaneously release large amounts of membrane vesicles into culture medium, from which they can be readily purified. For instance, the vesicles can be purified using the two stage size filtration process of ref. 152, comprising: (i) a first filtration step in which vesicles are separated from the bacteria based on their different sizes, with the vesicles passing into the filtrate; and (ii) a second filtration step in which the vesicles are retained in the retentate. The MltA mutation (down-regulation or knockout) has been used in ‘GMMA’ vaccines [153], and can conveniently be combined with further down regulation or knockout of in particular of at least one gene involved in rendering toxic the lipid A portion of LPS, particularly of ipxl1 and/or of at least one gene involved in the capsular polysaccharide synthesis or export, particularly of synX and/or ctrA genes.

A preferred meningococcal strain for a ‘GMMA’ (Generalized Module for Membrane Antigens) vaccine using this approach expresses a mutant v2 fHbp of the first, third or fifth aspect and/or a mutant v3 fHbp of the second, fourth or sixth aspect of the invention, and expression can be driven by strong promoters. Vesicles released by this strain include the mutant v2 and/or v3 fHbp proteins in immunogenic form, and administration of the vesicles can provide bactericidal antibody response as discussed in reference 153. The strain can also express a v1 fHbp, or a v1 fHbp can instead be provided as a separate recombinant protein in soluble form (and the v1 fHbp can be a wild-type or a mutant sequence e.g. mutated to disrupt its ability to bind to fH, as discussed above). The invention provides such strains, and also provides the vesicles which these strains release e.g. as purified from culture media after growth of the strains. A preferred v2 mutant for expression in these strains has a mutation at L123 and E240 (and optionally S32) as discussed herein, and a preferred v3 mutant for expression in these strains has a mutation at L126 and E243 (and optionally S32) as discussed herein. Thus vesicles prepared from meningococci expressing these v2 and v3 mutant fHbp sequences are particularly preferred immunogens for use in vaccines of the invention. A useful wild-type v2 sequence for mutagenesis in this way comprises SEQ ID NO: 35 or SEQ ID NO: 33 (comprising ΔG form SEQ ID NO: 34), and a useful wild-type v3 sequence for mutagenesis in this way comprises SEQ ID NO: 36.

Useful promoters for use in such strains include those disclosed in references 154 and 155. For instance, the promoter can be: (a) the promoters from a porin genes, preferably porA or porB, particularly from N. meningitidis; or (b) arRNA gene promoter (such as a 16S rRNA gene), particularly from N. meningitidis. Where a meningococcal porin promoter is used, it is preferably from porA, and even more particularly a -10 region from a meningococcal porA gene promoter, and/or a -35 region from a meningococcal porA gene promoter (preferably wherein the -10 region and the -35 region are separated by an intervening sequence of 12-20 nucleotides, and wherein the intervening sequence either contains no poly-G sequence or includes a poly-G sequence having no more than eight consecutive G nucleotides). Where a rRNA gene promoter is used, it can comprise more particularly (i) a -10 region from a meningococcal rRNA gene promoter and/or (ii) a -35 region from a meningococcal rRNA gene promoter. It is also possible to use a hybrid of (a) and (b), for instance to have a -10 region from a porA promoter and a -35 region from a rRNA promoter (which can be a consensus -35 region). A useful promoter can thus be a promoter which includes either (i) a -10 region from a (particularly meningococcal) rRNA gene and a -35 region from a (particularly meningococcal) porA gene, or (ii) a -10 region from a (particularly meningococcal) porA gene and a -35 region from a (particularly meningococcal) rRNA gene.

### General

The term “comprising” encompasses “including” as well as “consisting” e.g. a composition “comprising” X may consist exclusively of X or may include something additional e.g. X+Y. References to “comprising” (or “comprises”, etc.) may optionally be replaced by references to “consisting of” (or “consists of”, etc.).

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

“Sequence identity” is preferably determined by the Needleman-Wunsch global alignment algorithm [156], using default parameters (e.g. with Gap opening penalty=10.0, and with Gap extension penalty =0.5, using the EBLOSUM62 scoring matrix). This algorithm is conveniently implemented in the needle tool in the EMBOSS package [157]. Where the application refers to sequence identity to a particular SEQ ID, the identity should be calculated over the entire length of that SEQ ID.

After serogroup, meningococcal classification includes serotype, serosubtype and then immunotype, and the standard nomenclature lists serogroup, serotype, serosubtype, and immunotype, each separated by a colon e.g. B:4:P1.15:L3,7,9. Within serogroup B, some lineages cause disease often (hyperinvasive), some lineages cause more severe forms of disease than others (hypervirulent), and others rarely cause disease at all. Seven hypervirulent lineages are recognised, namely subgroups I, III and IV-1, ET-5 complex, ET-37 complex, A4 cluster and lineage 3. These have been defined by multilocus enzyme electrophoresis (MLEE), but multilocus sequence typing (MLST) has also been used to classify meningococci. The four main hypervirulent clusters are ST32, ST44, ST8 and ST11 complexes.

In general, the invention does not encompass the various fHbp sequences specifically disclosed in references 2, 3, 5, 6, 7, 158, 159, 160, 161, 162, 163, 164, and 165.

### EXAMPLES

### Example 1: Mutagenesis for fH binding

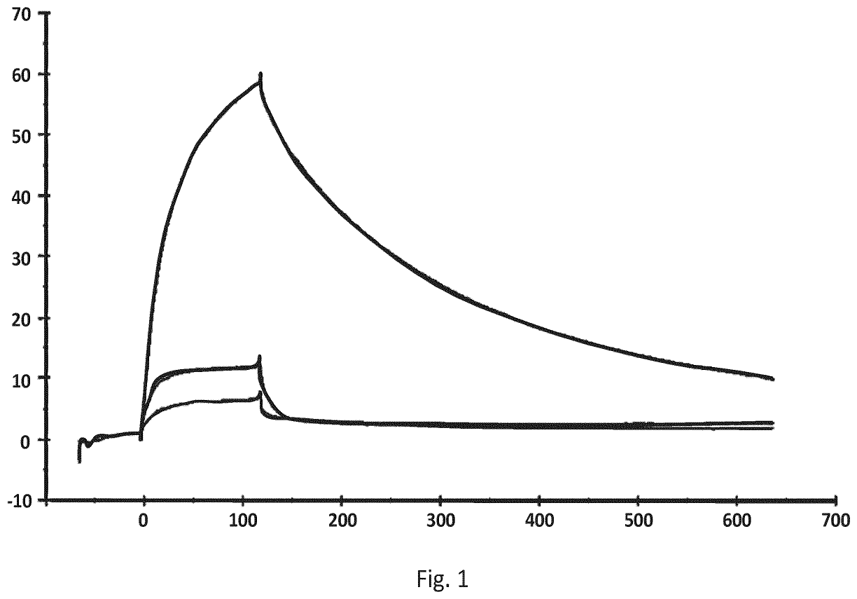

Wild-type v2 protein (SEQ ID NO:2) shows strong binding to fH when assessed by surface plasmon resonance (SPR) using immobilised human fH (FIG. 1, top line). To disrupt fHbp's ability to bind to fH, Glu-266 in v2 (E240 in SEQ ID NO: 5; corresponds to E248 in references 19 & 25) and Glu-274 in v3 (E243 in SEQ ID NO: 17) were mutated to Ala. The E266A mutation in v2 strongly reduced fH binding (FIG. 1, bottom line).

Similarly, the known ‘R41S’ mutation of v1 fHbp was introduced (SEQ ID NO: 52).

### Example 2: Mutagenesis for Stability Increase

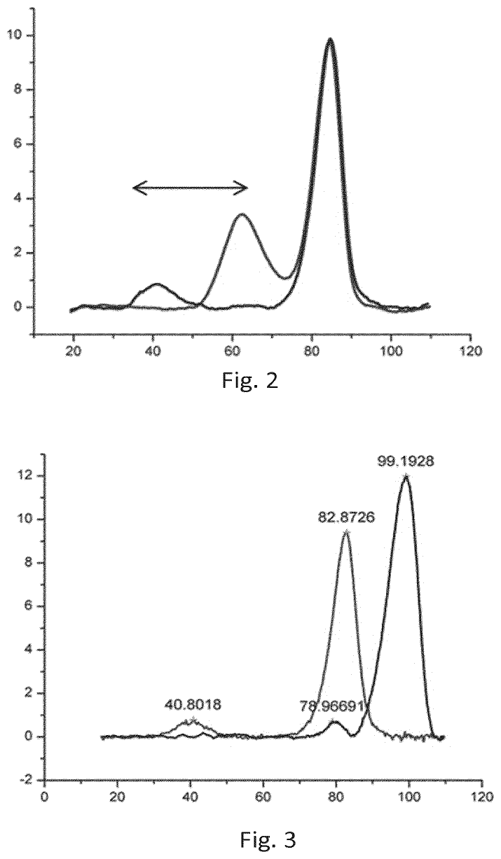

Both v2 and v3 fHbp are significantly less stable than v1, particularly in their N-terminal domains, and v2 is the least stable of the three variants. To improve stability in v2, two residues were mutated: Ser-58 of SEQ ID NO:2 (S32 in SEQ ID NO: 5) and Leu-149 of SEQ ID NO:2 (L123 in SEQ ID NO: 5) were mutated to Val and Arg, respectively. The mutant v2 protein (SEQ ID NO: 19) was analysed by DSC and, compared to the wild-type sequence SEQ ID NO:2, the Tₘ of the C-terminal domain was not affected by the mutation. TheTₘ of the N-terminus domain is ≥20° C. higher (FIG. 2, increase marked with arrow).The equivalent mutations have also been introduced into v3 (SEQ ID NO: 44).

Surprisingly, although the S58V and L149R mutations had been introduced to improve stability, and did indeed achieve this goal, FIG. 1 (middle line) shows that the mutant polypeptide (SEQ ID NO: 19) (even without the E266A mutation) also showed much reduced binding to fH. Furthermore, in a serum bactericidal assay this v2 mutant could compete for binding to human antibodies which had been raised against SEQ ID NO: 18:

| rSBA | Strain: Var 2.19 | Strain: Var 2.16 |
|---|---|---|
| Competitor | Rabbit 20-1 | Rabbit 20-1 |
| | 741(2-3-1) | 741(2-3-1) |
| None | 2048 | 4096 |
| 741 V2 S58/L149R | <16 | <16 |

The S58V/L149R stabilising mutation in v2 had a surprising impact on fH binding, so the effect of E266A on stability was also investigated. Unexpectedly, this mutation decreased the stability of the N-terminus domain, but increased stability of the C-terminus domain by ≥15° C. (from 83° C. up to 99° C., as shown in FIG. 3, compared to wild-type, thus suggesting a potential stabilisation of the beta barrel.

The effects of the individual S58V and L149R mutations on fH binding were studied in v3. Thus, numbered according to SEQ ID NO: 17, mutation S32V or L126R was introduced into the v3 sequence. These two mutants were compared to two different wild-type v3 sequences, and also to the ‘E313A’ mutant which is known to disrupt fH binding in v3 [23].

As shown inFIG. 6, both wild-type v3 bind fH (top two lines). The S58V mutation, which was designed to improve stability, reduced the SPR peak by about 2-fold. Most surprisingly, the L 149R mutation (again, designed to improve stability) reduced fH affinity to a similar level to the known E313A mutant (bottom two lines).

The S58V and L149R mutations in v3 were also studied by DSC, and were found to increase the N-terminal Tm by 5.5° C. (S58V) or by 6.7° C. (L149R). The Tm of each mutant was higher than seen in the v2 S58V/L149R double mutant. The L149R v3 mutant also showed a higher Tm value for its C-terminal domain, whereas there was almost no shift for the S58V v3 mutant.

### Example 3: Fusion Polypeptides

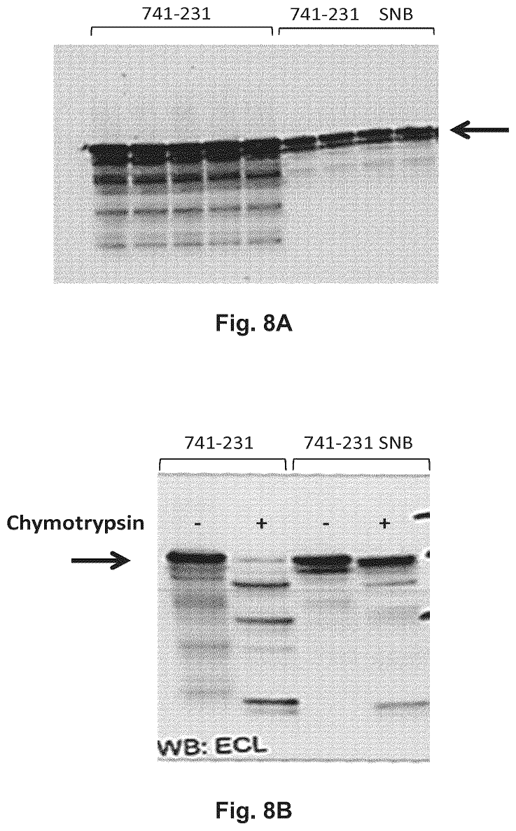

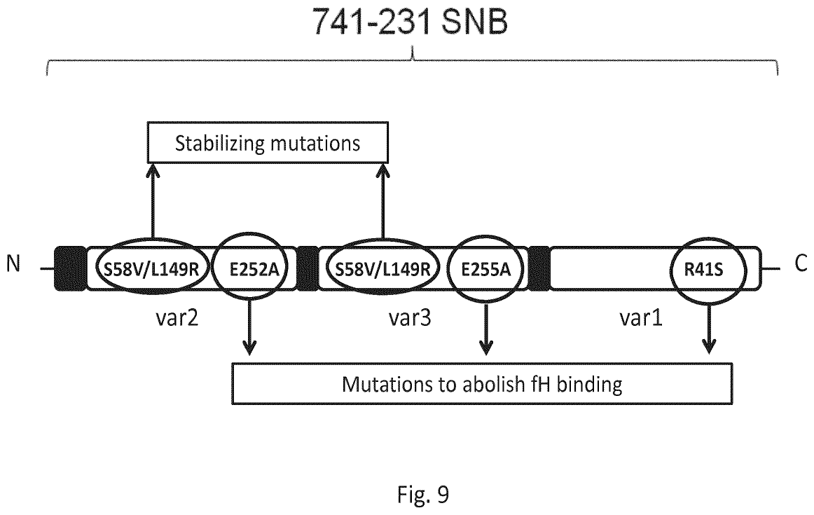

The mutations for stability and fHbp binding were combined into mutant forms of v2 (SEQ ID NO: 50) and v3 (SEQ ID NO: 51). These were fused with the mutant v1 sequence (SEQ ID NO:52) in the order v2-v3-v1 and were joined using linkers, to give SEQ ID NO: 27 (‘SNB’). Thus, compared to the three wild-type sequences, this fusion polypeptide includes a total of 7 point mutations (FIG. 9). The SNB fusion was compared to a ‘wild-type’ fusion polypeptide without these point mutations (SEQ ID NO: 18; SEQ ID NO: 36 from reference 163). E. coli extracts expressing both forms of the protein were probed by western blot, and degradation forms of the protein were much less visible using the stabilised non-binding forms of the fusion (SEQ ID NO: 27) (FIG. 8A and FIG. 8B).

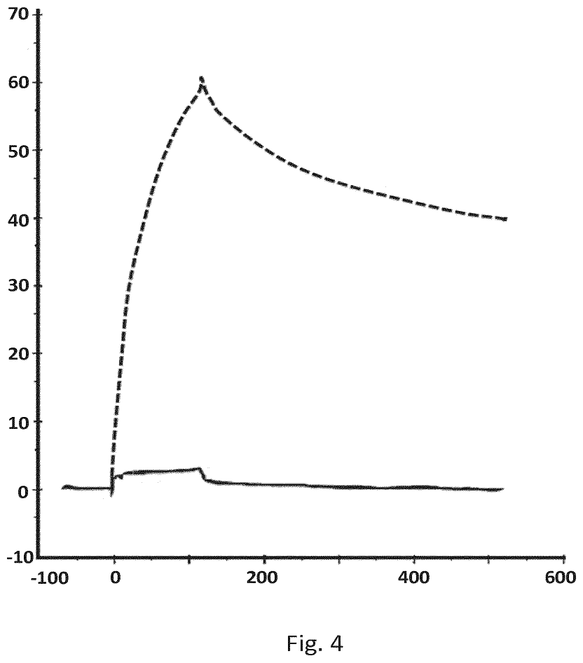

Binding of the SNB fusion to fH was investigated by SPR, and compared to the ‘wild-type’ fusion. FIG. 4 shows that the ‘wild-type’ fusion shows strong binding to fH (top line), whereas the SNB mutant does not interact significantly with fH (bottom line).

Stability of the two fusion polypeptides was investigated using DSC (FIG. 5A and FIG. 5B). The thermogram for the ‘wild-type’ fusion (FIG. 5A) did not include any N-terminal transition attributable to v3, suggesting that this domain was not correctly folded. In contrast, with the ‘SNB’ mutant the thermogram showed transitions for all 6 domains (3 each for N- and C-termini), indicating that they are all correctly folded (FIG. 5B).

Separately, the mutations for stability in v2 (SEQ ID NO: 45) and v3 (SEQ ID NO: 44) were fused with the ‘R41S’ mutant v1 sequence (SEQ ID NO:52) in the order v2-v3-v1 and were joined using linkers, to give SEQ ID NO: 29. Thus, compared to the three wild-type sequences, this fusion polypeptide includes a total of 5 point mutations.

The ability of non-fH binding forms of fHbp to elicits SBA titers was tested in transgenic (Tg) mice:

| | rSBA titers obtained against prototypic strains | | |
|---|---|---|---|
| Antigen | Var 1.1 | Var 2.16 | Var 3.42 |
| fHbp fusion | 1024* | 4096 | 8192 |
| SEQ ID NO: 18 | | | |
| fHbp fusion | 16384 | 32768 | >32768 |
| SEQ ID NO: 27 | | | |

These data indicate that non-binding forms of fHbp may be more immunogenic.

### Example 4: 3D Structures

Previously, the fHbp var.3 structure has been resolved only in complex with fH. For the v2 fHbp-fH complex, only the C-terminal domain of fHbp was detectable in previous studies.

Crystals of the V2 and V3 fHbp mutants were prepared as followed: Crystallization experiments were performed using a Gryphon crystallization robot (Art Robbins Instruments). X-ray diffraction data were collected at the Swiss Light Source (Paul Scherrer Institute, Villigen, Switzerland) beamline X06DA on a Pilatus 2M detector or collected on beamline BM30A of European Synchrotron Radiation Facility (ESRF), Grenoble, France. All diffraction data were processed with iMosfim, scaled with Aimless and crystallographic manipulations were carried out with the CCP4 package.

| fHbp Construct | Crystals | Structure (Å) |
|---|---|---|
| fHbp v2.1 S58V/L149R | YES | YES (1.7) |
| fHbp v3.1 S58V/L149R | YES | YES (3.3) |

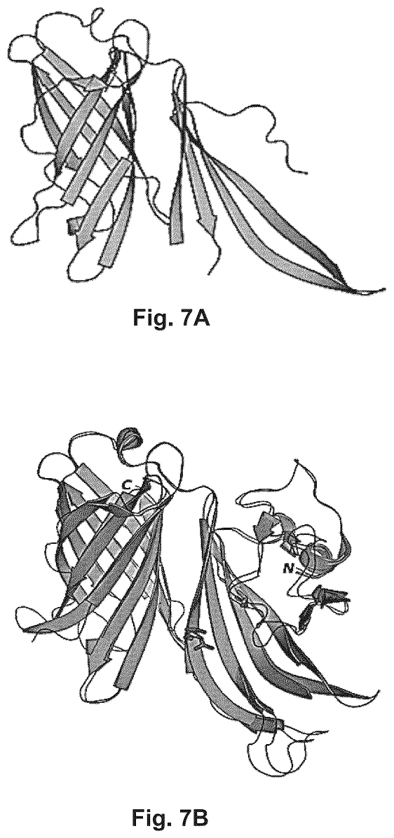

Stabilizing mutations potentiate structure determination of var.2 Nterminus and the X-ray structure of fHbp var.3 S58V has been solved in the absence of fH. fHbp var.2 and var.3 are characterized by a less stable folding in comparison with var.1. In line with this observation, the full length structure of fHbp var2 and var3 has been difficult to determine. Stabilization of protein translates into preservation of both structure and functionality, coupled to the establishment of a better thermodynamic equilibrium with the (micro)environment. As a result, protein stabilization often results in the obtainment of crystals suitable for structure determination. The S58V and L149R stabilized substitutions enabled determination of the entire fHbp var.3 crystal structure and the resolution of the segment 81-254 of fhbp var.2. By introducing stabilising mutations the almost complete structure of the N-terminal has been obtained in the absence of fH (FIG. 7A and FIG. 7B).

### Example 5: Surface Plasmon Resonance (SPR) Analysis

SPR was used to analyze the binding of 231 chimeric proteins to fH proteins. All SPR experiments were performed using a Biacore T200 instrument at 25° C. (GE Healthcare). In brief a carboxymethylated dextran sensor chip (CM-5; GE Healthcare) was prepared where similar densities (˜400-500 response units (RUs)) of 231 proteins were immobilized by amine coupling. The proteins immobilized were:
231 wt (SEQ ID NO: 18) purification MenB 547 (0.26 mg/ml) immobilized on Flow cell 2
231 S, comprising R41S, S58V and L149 for both v2 and v3, (SEQ ID NO: 29) purification MenB 532 (0.68 mg/ml) immobilized on Flow cell 3
231 SNB, comprising R41S, S58V and L149 for both v2 and v3, E252A and E255A (SEQ ID NO: 27) purification MenB 512 (0.78 mg/ml) immobilized on Flow cell 4

These proteins were diluted to 5 μg/ml in Acetate pH 5.5 and a standard amine coupling protocol was followed to reach target density. Flow cell 1 was prepared as the other Fes but no protein was used. Flow cell 1 was then used as reference cell and resulting signal was subtracted from the signal resulting from other flow cells. Running buffer contained 10 mM Hepes, 150 mM NaCl, 0.05% (vol/vol) P20 surfactant, pH 7.4 (HBS-P-GE-Healthcare). Then fH proteins were injected as a range of five injections of increasing analyte concentration with a 2 fold dilution (62.5 nM to 1 μM) for binding experiments. The following fH constructs were tested: factor H full length (Calbiochem) and factor H comprising only domains 6-7 (Schneider et al., Nature 458, 890-893) provided by C. Tang.

After each injection surfaces were then regenerated with an injection of 20 seconds of 10 mM glycine pH 1.7. A blank injection of buffer only was subtracted from each curve, and reference sensorgrams were subtracted from experimental sensorgrams to yield curves representing specific binding. The data shown are representative of two independent experiments. SPR data were analyzed using the Biacore T200 Evaluation software (GE Healthcare). Resulting sensorgrams were fitted with the 1:1 Langmuir binding model, including a term to account for potential mass transfer, to obtain the individual kon and koff kinetic constants; the individual values were then combined to derive the single averaged KD values (KD=koff/kon) reported. Steady-state analysis was also used to obtain thermodynamic dissociation constants (KD) at pH 7.4. Results of Titration with injections of fH domains 6-7 are shown below:

| | | | | K_{D} | K_{D} from | | |
|---|---|---|---|---|---|---|---|
| | fH | Kₒₙ | K_{off} | from 1:1 | steady state | Ration K_{D} wt/x | |
| Fusion | prot. | (1/Ms) | (1/s) | fitting (M) | analysis (M) | (residual binding) | |
| 231 WT | fH 67 | 6.4E+5 | 0.008 | 1.24E−8 | 2.1E−8 | 1 | (wt/wt) |
| 231 S | fH 67 | 7.1E+5 | 0.11 | 1.58E−7 | 2.8E−7 | 0.076 | (wt/S) |
| 231 SNB | fH 67 | 2.8E+5 | 0.29 | 1.05E−6 | 2.0E−6 | 0.011 | (wt/SNB) |

From binding tests a strong reduction of at least 90% of the binding to fH was observed for the 231 S protein compared to 231 wt and of at least 98% for the 231 SNB protein compared to 231 wt.

Results of the titration with fH full length are provided below:

| | fH | Kₒₙ | K_{off} | K_{D} from 1:1 | Ration K_{D} wt/x | |
|---|---|---|---|---|---|---|
| Fusion | prot. | (1/Ms) | (1/s) | fitting (M) | (residual binding) | |
| 231 WT | fH f.1. | 1.1E+4 | 0.003 | 3.1E−7 | 1 | (wt/wt) |
| 231 S | fH f.1. | 2.1E+4 | 0.064 | 3.1E−6 | 0.1 | (wt/S) |
| 231 SNB | fH f.1. | 2.7E+3 | 0.06 | 2.1E−5 | 0.015 | (wt/SNB) |

From both binding tests we observe a strong reduction of at least 90% of the binding to fH for the 231 S protein compared to 231 wt and of 98% for the 231 SNB protein compared to 231 wt.

It will be understood that the invention is described above by way of example only and modifications may be made whilst remaining within the scope and spirit of the invention.

### REFERENCES

[1] WO99/57280.
[2] Masignani et al. (2003) J Exp Med 197:789-799.
[3] Welsch et al. (2004) J Immunol 172:5605-15.
[4] Hou et al. (2005) J Infect Dis 192(4):580-90.
[5] WO03/063766.
[6] Fletcher et al. (2004) Infect Immun 72:2088-2100.
[7] Zhu et al. (2005) Infect Immun 73(10):6838-45.
[8] Cendron et al. (2011) Acta Crystallogr Sect F Struct Biol Cryst Commun. 67:531-5.
[9] Mascioni et al. (2009) J Biol Chem 284:8738-46.
[10] Pizza et al. (2008) Vaccine 26 Suppi 8:I46-8.
[11] Malito et al. (2013) PNAS USA 110:3304-9.
[12] Marshall et al. (2012) Pediatr Infect Dis J31:1061-8.
[13] McNeil et al. (2013) Microbiol Mol Biol Rev 77:234-52.
[14] Serruto et al. (2012) Vaccine 30 Suppl 2: B87-97.
[15] Scarselli et al. (2011) Sci Transl Med 3:91ra62.
[16] WO2011/051893.
[17] WO2010/046715.
[18] Schneider et al. (2009) Nature 458:890-5.
[19] WO2011/126863.
[20] Beernink et al. (2010) Clin Vaccine Immunol 17:1074-8.
[21] Beernink et al. (2011) J Immunol 186:3606-14.
[22] Rossi et al. (2013) Vaccine 31:5451-7.
[23] van der Veen et al. (2014) Infect Immun PMID 24379280.
[24] Johnson et al. (2012) PLoS Pathogen 8:e1002981.
[25] Pajon et al. (2012) Infect Immun 80:2667-77.
[26] Granoff et al. (2013) Clin Vaccine Immunol 20:1099-107.
[27] WO2014/030003.
[28] Beernink et al. (2008) Infect Immun 76:4232-40.
[29] Scarselli et al. (2009) J Mol Biol 386:97-108.
[30] Giuntini et al. (2012) PLoS One 7:e34272.
[31] Vu et al. (2012) Sci Rep 2:341.
[32] Faleri et al. (2013) FASEB Jfj.13-239012.
[33] Johnson (2013) Arch Biochem Biophys 531:100-9.
[34] Bruylants et al. (2005) Current Medicinal Chemistry 12:2011-20.
[35] Veggi et al. (2012) Biochemistry 51:9384-93.
[36] Pizza et al. (2000) Science 287:1816-1820.
[37] WO2007/028408.
[38] http://pubm!st.org/neisseria/
[39] Budroni et al. (2011) PNAS USA 108:4494-99.
[40] Goldschneider et al. (1969) J. Exp. Med. 129:1307-26.
[41] Santos et al. (2001) Clinical and Diagnostic Laboratory Immunology 8:616-23.
[42] Frasch et al. (2009) Vaccine 27S:B112-6.
[43] Gennaro (2000) Remington: The Science and Practice of Pharmacy. 20th edition, ISBN: 0683306472.
[44] WO03/009869.
[45] Vaccine Design . . . (1995) eds. Powell & Newman. ISBN: 030644867X. Plenum.
[46] Tettelin et al. (2000) Science 287:1809-1815.
[47] WO00/66741.
[48] Martin et al. (1997) J Exp Med 185(7):1173-83.
[49] WO96/29412.
[50] Perkins-Balding et al. (2003) Microbiology 149:3423-35.
[51] WO01/55182.
[52] WO01/38350.
[53] WO00/23595.
[54] Giuliani et al. (2006) Proc Natl Acad Sci USA. 103:10834-9.
[55] WO2004/032958.
[56] Costantino et al. (1992) Vaccine 10:691-698.
[57] Costantino et al. (1999) Vaccine 17:1251-1263.
[58] WO03/007985.
[59] Watson (2000) Pediatr Infect Dis J 19:331-332.
[60] Rubin (2000) Pediatr Clin North Am 47:269-285, v.
[61] Jedrzejas (2001) Microbiol Mol Biol Rev 65:187-207.
[62] Bell (2000) Pediatr Infect Dis J 19:1187-1188.
[63] Iwarson (1995) APMIS 103:321-326.
[64] Gerlich et al. (1990) Vaccine 8 Suppl:S63-68 & 79-80.
[65] Vaccines (1988) eds. Plotkin & Mortimer. ISBN 0-7216-1946-0.
[66] Del Guidice et al. (1998) Molecular Aspects of Medicine 19:1-70.
[67] Gustafsson et al. (1996) N. Engl. J. Med. 334:349-355.
[68] Rappuoli et al. (1991) TIBTECH 9:232-238.
[69] Sutter et al. (2000) Pediatr Clin North Am 47:287-308.
[70] Zimmerman & Spann (1999) Am Fam Physician 59:113-118, 125-126.
[71] McMichael (2000) Vaccine 19 Suppl 1:S101-107.
[72] Schuchat (1999) Lancet 353(9146):51-6.
[73] WO02/34771.
[74] Dale (1999) Infect Dis Clin North Am 13:227-43, viii.
[75] Ferretti et al. (2001) PNAS USA 98: 4658-4663.
[76] Kuroda et al. (2001) Lancet 357(9264):1225-1240; see also pages 1218-1219.
[77] Jones (2001) Curr Opin Investig Drugs 2:47-49.
[78] Ravenscroft et al. (1999) Vaccine 17:2802-2816.
[79] WO03/080678.
[80] Research Disclosure, 453077 (January 2002).
[81] EP-A-0372501.
[82] EP-A-0378881.
[83] EP-A-0427347.
[84] WO93/17712.
[85] WO94/03208.
[86] WO98/58668.
[87] EP-A-0471177.
[88] WO91/01146.
[89] Falugi et al. (2001) Eur J Immunol 31:3816-3824.
[90] Baraldo et al. (2004) Infect Immun 72(8):4884-7.
[91] EP-A-0594610.
[92] Ruan et al. (1990) J Immunol 145:3379-3384.
[93] WO00/56360.
[94] Kuo et al. (1995) Infect Immun 63:2706-13.
[95] Michon et al. (1998) Vaccine. 16:1732-41.
[96] WO02/091998.
[97] WO01/72337.
[98] WO00/61761.
[99] WO00/33882
Lees et al. (1996) Vaccine 14:190-198.
WO95/08348.
U.S. Pat. No. 4,882,317
U.S. Pat. No. 4,695,624
Porro et al. (1985) Mol Immunol 22:907-919.s
EP-A-0208375
WO00/10599
Gever et al. Med. Microbiol. Immunol, 165: 171-288 (1979).
U.S. Pat. No. 4,057,685.
U.S. Pat. Nos. 4,673,574; 4,761,283; 4,808,700.
U.S. Pat. No. 4,459,286.
U.S. Pat. No. 4,965,338
U.S. Pat. No. 4,663,160.
U.S. Pat. No. 4,761,283
U.S. Pat. No. 4,356,170
WO02/09643.
Katial et al. (2002) Infectlmmun 70:702-707.
WOO1/52885.
European patent 0301992.
Bjune et al. (1991) Lancet 338(8775):1093-1096.
Frasch et al. (2001) chapter 7 of Methods in Molecular Medicine, volume 66 (‘Meningococcal Vaccines: Methods and Protocols’, eds. Pollard & Maiden).
Fukasawa et al. (1999) Vaccine 17:2951-2958.
WO02/09746.
Rosenqvist et al. (1998) Dev. Biol. Stand. 92:323-333.
European patent 0011243.
Fredriksen et al. (1991) NIPHAnn. 14(2):67-80.
WO01/91788.
WO2004/019977.
U.S. Pat. No. 6,558,677.
WO01/09350.
European patent 0449958.
EP-A-0996712.
EP-A-0680512.
WO02/062378.
WO99/59625.
U.S. Pat. No. 6,180,111.
WO01/34642.
Peeters et al. (1996) Vaccine 14:1008-1015.
Vermont et al. (2003) Infect Immun 71:1650-1655.
WO2004/014417.
WO2005/004908.
WO2006/081259.
WO98/56901.
Claassen et al. (1996) 14(10):1001-8.
WO99/10497.
Steeghs et al. (2001) The EMBO Journal 20:6937-6945.
Fisseha et al. (2005) Infect Immun 73:4070-80.
WO00/25811.
WO2004/015099.
WO2004/046177.
WO2006/046143.
Adu-Bobie et al. (2004) Infect Immun 72:1914-19.
WO2011/036562.
Koeberling et al. (2014) Vaccine 32:2688-95.
WO2013/033398.
WO2013/113917.
Needleman & Wunsch (1970) J. Mol. Biol. 48, 443-453.
Rice et al. (2000) Trends Genet 16:276-277.
WO01/64920.
WO03/020756.
WO2004/048404.
WO2004/094596
WO2006/024954.
WO2007/060548.
WO2009/104097.
WO2013/132452.

1-18. (canceled)

19. A composition comprising:
a meningococcal factor H binding protein (fHbp) having an amino acid sequence with at least 80% sequence identity to SEQ ID NO: 47, wherein the amino acid sequence, relative to the sequence of SEQ ID NO: 47, at residue 123 is not leucine.

20. The composition of claim 19, wherein the amino acid sequence, relative to the sequence of SEQ ID NO: 47, has the substitution L123R.

21. The composition of claim 20, wherein the amino acid sequence, relative to the sequence of SEQ ID NO: 47, at residue 32 is serine.

22. The composition of claim 21, wherein the amino acid sequence, relative to the sequence of SEQ ID NO: 47, at residue 240 is glutamate.

23. The composition of claim 19, further comprising a pharmaceutically acceptable carrier.

24. The composition of claim 19, further comprising a NadA antigen.

25. The composition of claim 19, further comprising outer membrane vesicles (OMVs).

26. The composition of claim 19, further comprising an adjuvant.

27. The composition of claim 19, further comprising a second meningococcal factor H binding protein (fHbp).

28. The composition of claim 27, further comprising a fusion polypeptide comprising (i) a polypeptide as defined in claim 19; and (ii) the second fHbp.

29. A composition comprising:
a meningococcal factor H binding protein (fHbp) having an amino acid sequence with at least 80% sequence identity to SEQ ID NO: 54, wherein the amino acid sequence, relative to the sequence of SEQ ID NO: 54, at residue 123 is arginine.

30. The composition of claim 29, wherein the amino acid sequence, relative to the sequence of SEQ ID NO: 54, at residue 32 is serine.

31. The composition of claim 30, wherein the amino acid sequence, relative to the sequence of SEQ ID NO: 54, at residue 240 is glutamate.

32. The composition of claim 31, further comprising a pharmaceutically acceptable carrier.

33. The composition of claim 32, further comprising a NadA antigen.

34. The composition of claim 33, further comprising outer membrane vescicles (OMVs).

35. The composition of claim 34, further comprising a second meningococcal factor H binding protein (fHbp).

36. The composition of claim 35, further comprising an adjuvant.

37. A method of raising an antibody response against meningococcus in a human, comprising administering to said human the composition according to claim 19.

38. A method of preventing or treating meningitis comprising administering to a human the composition of claim 19.


SEQUENCE LISTING (60 sequences)

SEQ ID NO: 1 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 5 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 6 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 7 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 8 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 9 - protein, 792 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYNHHALGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPAKNLTLRAGVYNLFNRKYTTWDSLRGLYSYSTTNAVDRDGKGLDRYRAPGRNYAVSLEWKF

SEQ ID NO: 10 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 11 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 12 - protein, 797 aa, Neisseria meningitidis
MKLKQIASALMMLGISPLALADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSNQFNEQKFAQDMEKVTDFYQNNGYFDFRILDTDIQTNEDKTKQTIKITVHEGGRFRWGKVSIEGDTNEVPKAELEKLLTMKPGKWYERQQMTAVLGEIQNRMGSAGYAYSEISVQPLPNAETKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDVYGKAFDPRKASTSIKQYKTTTAGAGIRMSVPVTEYDRVNFGLVAEHLTVNTYNKAPKHYADFIKKYGKTDGTDGSFKGWLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGKTYDDNSSSATGGRVQNIYGAGNTHKSTFTNELRYSAGGAVTWLSPLGPMKFSYAYPLKKKPEDEIQRFQFQLGTTF

SEQ ID NO: 13 - protein, 180 aa, Neisseria meningitidis
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQR

SEQ ID NO: 14 - protein, 644 aa, Neisseria meningitidis
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 15 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 16 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 17 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 18 - protein, 776 aa, synthetic construct
  REGION 1..776: note = Description of Artificial Sequence: Synthetic polypeptide
MGPDSDRLQQRRVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGPDSDRLQQRRVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 19 - protein, 273 aa, synthetic construct
  REGION 1..273: note = Description of Artificial Sequence: Synthetic polypeptide
MNRTAFCCLSLTAALILTACSSGGGGVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 20 - protein, 6 aa, synthetic construct
  REGION 1..6: note = Description of Artificial Sequence: Synthetic peptide
GSGGGG

SEQ ID NO: 21 - protein, 11 aa, synthetic construct
  REGION 1..11: note = Description of Artificial Sequence: Synthetic peptide
GPDSDRLQQRR

SEQ ID NO: 22 - protein, 13 aa, synthetic construct
  REGION 1..13: note = Description of Artificial Sequence: Synthetic peptide
GSGPDSDRLQQRR

SEQ ID NO: 23 - protein, 13 aa, synthetic construct
  REGION 1..13: note = Description of Artificial Sequence: Synthetic peptide
GKGPDSDRLQQRR

SEQ ID NO: 24 - protein, 12 aa, synthetic construct
  REGION 1..12: note = Description of Artificial Sequence: Synthetic peptide
MGPDSDRLQQRR

SEQ ID NO: 25 - skipped

SEQ ID NO: 26 - protein, 8 aa, synthetic construct
  REGION 1..8: note = Description of Artificial Sequence: Synthetic peptide
LEHHHHHH

SEQ ID NO: 27 - protein, 769 aa, synthetic construct
  REGION 1..769: note = Description of Artificial Sequence: Synthetic polypeptide
MGPDSDRLQQRRVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 28 - protein, 757 aa, synthetic construct
  REGION 1..757: note = Description of Artificial Sequence: Synthetic polypeptide
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 29 - protein, 769 aa, synthetic construct
  REGION 1..769: note = Description of Artificial Sequence: Synthetic polypeptide
MGPDSDRLQQRRVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 30 - protein, 757 aa, synthetic construct
  REGION 1..757: note = Description of Artificial Sequence: Synthetic polypeptide
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 31 - protein, 247 aa, synthetic construct
  VARIANT 32: note = MOD_RES - Any amino acid
  VARIANT 123: note = MOD_RES - Any amino acid except Leu
  REGION 1..247: note = Description of Artificial Sequence: Synthetic polypeptide
VAADIGAGLADALTAPLDHKDKSLQSLTLDQXVRKNEKLKLAAQGAEKTYGNGDSLNTGKLKNDKVSRFDFIRQIEVDGQLITLESGEFQIYKQDHSAVVALQIEKINNPDKIDSLINQRSFXVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 32 - protein, 250 aa, synthetic construct
  VARIANT 32: note = MOD_RES - Any amino acid
  VARIANT 126: note = MOD_RES - Any amino acid except Leu
  REGION 1..250: note = Description of Artificial Sequence: Synthetic polypeptide
VAADIGTGLADALTAPLDHKDKGLKSLTLEDXIPQNGTLTLSAQGAEKTFKAGDKDNSLNTGKLKNDKISRFDFVQKIEVDGQTITLASGEFQIYKQNHSAVVALQIEKINNPDKTDSLINQRSFXVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 33 - protein, 273 aa, Neisseria meningitidis
MNRTAFCCLSLTTALILTACSSGGGGVAADIGARLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 34 - protein, 247 aa, Neisseria meningitidis
VAADIGARLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 35 - protein, 247 aa, Neisseria meningitidis; identical to SEQ ID NO: 34

SEQ ID NO: 36 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAELKADEKSHAVILGDTRYGGEEKGTYHLALFGDRAQEIAGSATVKIREKVHEIGIAGKQ

SEQ ID NO: 37 - protein, 250 aa, synthetic construct; identical to SEQ ID NO: 32
  VARIANT 32: note = MOD_RES - Any amino acid except Ser
  VARIANT 126: note = MOD_RES - Any amino acid except Leu
  REGION 1..250: note = Description of Artificial Sequence: Synthetic polypeptide

SEQ ID NO: 38 - protein, 247 aa, synthetic construct
  VARIANT 32: note = MOD_RES - Any amino acid except Ser
  VARIANT 123: note = MOD_RES - Any amino acid except Leu
  VARIANT 240: note = MOD_RES - Any amino acid except Glu
  REGION 1..247: note = Description of Artificial Sequence: Synthetic polypeptide
VAADIGAGLADALTAPLDHKDKSLQSLTLDQXVRKNEKLKLAAQGAEKTYGNGDSLNTGKLKNDKVSRFDFIRQIEVDGQLITLESGEFQIYKQDHSAVVALQIEKINNPDKIDSLINQRSFXVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHXIGIAGKQ

SEQ ID NO: 39 - protein, 262 aa, synthetic construct
  VARIANT 32: note = MOD_RES - Any amino acid except Ser
  VARIANT 123: note = MOD_RES - Any amino acid except Leu
  VARIANT 240: note = MOD_RES - Any amino acid except Glu
  REGION 1..262: note = Description of Artificial Sequence: Synthetic polypeptide
CSSGGGGSGGGGVAADIGTGLADALTAPLDHXDKGLKSLTLEDSIPQNGTLTLSAQGAEKTFKAGDKDNSLNTGKLKNDKISRFDFVQKIEVDGQTITLASGEFQIYKQNHSAVVALQIEKIXNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQXIAGSATVKIGEKVHEIGIAGKQ

SEQ ID NO: 40 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 41 - protein, 250 aa, synthetic construct
  REGION 1..250: note = Description of Artificial Sequence: Synthetic polypeptide
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 42 - protein, 250 aa, synthetic construct
  REGION 1..250: note = Description of Artificial Sequence: Synthetic polypeptide
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 43 - protein, 250 aa, synthetic construct
  REGION 1..250: note = Description of Artificial Sequence: Synthetic polypeptide
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 44 - protein, 250 aa, synthetic construct
  REGION 1..250: note = Description of Artificial Sequence: Synthetic polypeptide
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 45 - protein, 247 aa, synthetic construct
  REGION 1..247: note = Description of Artificial Sequence: Synthetic polypeptide
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 46 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 47 - protein, 247 aa, synthetic construct; identical to SEQ ID NO: 38
  VARIANT 32: note = MOD_RES - Any amino acid
  VARIANT 123: note = MOD_RES - Any amino acid except Leu
  VARIANT 240: note = MOD_RES - Any amino acid except Glu
  REGION 1..247: note = Description of Artificial Sequence: Synthetic polypeptide

SEQ ID NO: 48 - protein, 250 aa, synthetic construct
  VARIANT 32: note = MOD_RES - Any amino acid
  VARIANT 126: note = MOD_RES - Any amino acid except Leu
  VARIANT 243: note = MOD_RES - Any amino acid except Glu
  REGION 1..250: note = Description of Artificial Sequence: Synthetic polypeptide
VAADIGTGLADALTAPLDHKDKGLKSLTLEDXIPQNGTLTLSAQGAEKTFKAGDKDNSLNTGKLKNDKISRFDFVQKIEVDGQTITLASGEFQIYKQNHSAVVALQIEKINNPDKTDSLINQRSFXVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHXIGIAGKQ

SEQ ID NO: 49 - protein, 248 aa, synthetic construct
  VARIANT 34: note = MOD_RES - Any amino acid except Arg
  REGION 1..248: note = Description of Artificial Sequence: Synthetic polypeptide
VAADIGAGLADALTAPLDHKDKGLQSLTLDQSVXKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 50 - protein, 247 aa, synthetic construct
  REGION 1..247: note = Description of Artificial Sequence: Synthetic polypeptide
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQ

SEQ ID NO: 51 - protein, 250 aa, synthetic construct; identical to SEQ ID NO: 43
  REGION 1..250: note = Description of Artificial Sequence: Synthetic polypeptide

SEQ ID NO: 52 - protein, 248 aa, synthetic construct
  REGION 1..248: note = Description of Artificial Sequence: Synthetic polypeptide
VAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 53 - protein, 247 aa, synthetic construct
  REGION 1..247: note = Description of Artificial Sequence: Synthetic polypeptide
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHAIGIAGKQ

SEQ ID NO: 54 - protein, 247 aa, synthetic construct
  REGION 1..247: note = Description of Artificial Sequence: Synthetic polypeptide
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 55 - protein, 250 aa, synthetic construct
  REGION 1..250: note = Description of Artificial Sequence: Synthetic polypeptide
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHAIGIAGKQ

SEQ ID NO: 56 - protein, 250 aa, synthetic construct
  REGION 1..250: note = Description of Artificial Sequence: Synthetic polypeptide
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 57 - protein, 250 aa, synthetic construct; identical to SEQ ID NO: 48
  VARIANT 32: note = MOD_RES - Any amino acid except Ser
  VARIANT 126: note = MOD_RES - Any amino acid except Leu
  VARIANT 243: note = MOD_RES - Any amino acid except Glu
  REGION 1..250: note = Description of Artificial Sequence: Synthetic polypeptide

SEQ ID NO: 58 - protein, 10 aa, synthetic construct
  VARIANT 3..10: note = May be absent
  REGION 1..10: note = misc_feature - Variant residues given in the sequence have no preference with respect to those in the annotations for variant positions
  REGION 1..10: note = Description of Artificial Sequence: Synthetic peptide
GGGGGGGGGG

SEQ ID NO: 59 - protein, 10 aa, synthetic construct
  VARIANT 3..10: note = May be absent
  REGION 1..10: note = misc_feature - Variant residues given in the sequence have no preference with respect to those in the annotations for variant positions
  REGION 1..10: note = Description of Artificial Sequence: Synthetic peptide
HHHHHHHHHH

SEQ ID NO: 60 - protein, 10 aa, synthetic construct
  VARIANT 4..10: note = May be absent
  REGION 1..10: note = misc_feature - Variant residues given in the sequence have no preference with respect to those in the annotations for variant positions
  REGION 1..10: note = Description of Artificial Sequence: Synthetic peptide
HHHHHHHHHH